(19) United States
(12) Patent Application Publication
(10) Pub. No.: US 2025/0163114 A1
(43) Pub. Date: May 22, 2025

(54) TREATMENT OF AMYOTROPHIC LATERAL SCLEROSIS

(71) Applicant: Brandeis University, Waltham, MA (US)

(72) Inventors: Greg Petsko, New York, NY (US); Dagmar Ringe, Cambridge, MA (US); Shulin Ju, Beavercreek, OH (US)

(73) Assignee: Brandeis University, Waltham, MA (US)

(21) Appl. No.: 19/024,159

(22) Filed: Jan. 16, 2025

Related U.S. Application Data
(63) Continuation of application No. 17/721,715, filed on Apr. 15, 2022.
Application No. 17/721,715 is a continuation of application No. 16/739,561, filed on Jan. 10, 2020, now Pat. No. 11,332,504.
Application No. 16/739,561 is a continuation of application No. 14/434,737, filed on Apr. 9, 2015, now Pat. No. 10,533,038.
(60) Provisional application No. 61/712,322, filed on Oct. 11, 2012.

(51) Int. Cl. C07K 14/47; A01K 67/0275; A61K 9/00; A61K 31/7088; A61K 31/7105; A61K 31/711; A61K 31/7115; A61K 31/7125; A61K 35/12; A61K 38/00; A61K 38/17; A61K 48/00; A61P 3/00; C12N 15/85; C12Q 1/02; C12Q 1/6883; G01N 33/50

(52) U.S. Cl. CPC C07K 14/47; A61K 9/0019; A61K 38/17; C12Q 1/025; G01N 33/5058; A01K 67/0275; A01K 2207/05; A01K 2207/10; A01K 2207/20; A01K 2217/206; A01K 2267/03; A01K 2267/0306; A01K 2267/0318; A01K 2267/0393; A61K 31/7088; A61K 31/7105; A61K 31/711; A61K 31/7115; A61K 31/7125; A61K 35/12; A61K 38/00; A61K 38/1709; A61K 48/00; A61K 48/005; A61K 48/0066; A61K 2300/00; A61P 3/00; C12N 15/85; C12Q 1/6883; C12Q 2600/106; G01N 33/5023; G01N 2333/47; G01N 2500/10; G01N 2800/56

(57) ABSTRACT

Nonsense-mediated mRNA decay (NMD) polypeptides, nucleic acids encoding NMD polypeptides, and methods of using such polypeptides and nucleic acids in the treatment of ALS and in screening for agents for the treatment of ALS are described.

### CROSS REFERENCE TO RELATED APPLICATIONS

The present application is a continuation of U.S. application Ser. No. 17/721,715, filed Apr. 15, 2022, which is a continuation of U.S. application Ser. No. 16/739,561 (now U.S. Pat. No. 11,332,504), filed Jan. 10, 2020, which is a continuation of U.S. application Ser. No. 14/434,737 (now U.S. Pat. No. 10,533,038), filed Apr. 9, 2015, which is a national phase application under 35 U.S.C. § 371 of PCT International Application No. PCT/US2013/063858, filed Oct. 8, 2013, which claims the benefit of U.S. provisional application Ser. No. 61/712,322, filed on Oct. 11, 2012, the contents of each which are herein incorporated by reference in their entirety.

### SEQUENCE LISTING

The Instant Application contains a Sequence Listing which has been submitted electronically in XML format and is hereby incorporated by reference in its entirety. Said XML copy, created on Jan. 15, 2025, is named “SEQ_LIST—107648053.xml” and is 49,832 bytes in size. The Sequence Listing does not go beyond the disclosure in the application as filed.

### BACKGROUND

Amyotrophic lateral sclerosis (ALS, also called Lou Gehrig's disease) is a relentlessly progressive, fatal neurodegenerative disease with a prevalence of about 5 people out of 100,000 each year and an average age of onset of about 60 years. Patients with ALS suffer from degeneration of motor neurons in the brain and spinal cord, which leads to progressive muscular weakness. ALS accounts for about 1/300 to 1/400 of all deaths, which means that about 1,000,000 people now alive in the United States will develop ALS. Death typically occurs 3-5 years after disease onset, due to respiratory paralysis. There is no effective treatment for the disease; the only approved ALS drug (riluzole) extends the lifespan of some ALS patients by only about 3 months. Thus, there remains a need for new therapeutic approaches for treatment of ALS.

### SUMMARY

The present disclosure encompasses the surprising discovery that agents involved in nonsense-mediated mRNA decay (NMD) can protect neuronal cells from damage associated with TDP-43 or FUS/TLS. The present invention therefore provides NMD agents for use in medicine, and specifically in treatment or prevention (e.g., delay of onset) of certain neurological disorders including specifically amyotrophic lateral sclerosis (ALS). For example, in various aspects, the present disclosure provides methods of reducing FUS/TLS or TDP-43 toxicity in a neuronal cell or glial cell suffering from or susceptible to such toxicity, comprising providing to the cell (e.g., in vitro or in vivo) a therapeutically effective amount of an NMD polypeptide, thereby reducing the FUS/TLS or TDP-43 toxicity in the cell. In some embodiments, the step of providing comprises administering a composition comprising the NMD polypeptide, a nucleic acid encoding the NMD polypeptide, and/or an activator of the NMD polypeptide. In some embodiments, the NMD polypeptide is a UPF1, UPF2, UPF3, SMG1, SMG5, SMG6, or SMG7 polypeptide. In some embodiments, the cell is a human neuronal cell or a human glial cell.

In various aspects, the present disclosure provides methods of treating a disease, disorder or condition associated with FUS/TLS or TDP-43 toxicity, comprising administering to a subject suffering from or susceptible to the disease, disorder or condition a therapeutically effective amount of an NMD polypeptide, a nucleic acid encoding an NMD polypeptide, and/or an activator or an NMD polypeptide, thereby treating the disease, disorder or condition. In some embodiments, the therapeutically effective amount is correlated with a statistically significant probability of reducing FUS/TLS or TDP-43 toxicity in a neuronal cell or a glial cell. In some embodiments, the therapeutically effective amount is correlated with a statistically significant probability of enhancing mRNA processing in a neuronal cell or a glial cell. In some embodiments, the disease, disorder or condition is not associated with SODI toxicity. In some embodiments, the NMD polypeptide, nucleic acid encoding the NMD polypeptide, and/or the activator of the NMD polypeptide is administered into the CNS of the subject, such as by intrathecal injection.

In various aspects, the present disclosure provides methods of treating ALS in a human subject, comprising: administering to a subject suffering from or susceptible to ALS a therapeutically effective amount of an NMD polypeptide, thereby treating the ALS in the subject. In some embodiments, the therapeutically effective amount is correlated with a statistically significant probability of reducing toxicity in a human neuronal cell or a human glial cell. In some embodiments, the toxicity is FUS/TLS or TDP-43 toxicity. In some embodiments, the toxicity is not SOD1 toxicity. In some embodiments, the therapeutically effective amount is correlated with a statistically significant probability of enhancing mRNA processing in a human neuronal cell or a human glial cell.

In various aspects, the present disclosure provides methods of identifying an agent useful in the treatment of ALS, comprising: contacting a population of neuronal cells or glial cells that are suffering from or susceptible to FUS/TLS or TDP-43 toxicity with a test agent; determining a number of viable cells in the population after the contacting step; and comparing the number of viable cells to a control; wherein a test agent that increases the number of viable cells relative to the control is identified as an agent useful in the treatment of ALS. In some embodiments, the neuronal cells or the glial cells are transfected with a nucleic acid encoding FUS/TLS or TDP-43.

In various aspects, the present disclosure provides methods of identifying an agent useful in the treatment of ALS, comprising: contacting a population of neuronal cells or glial cells that are suffering from or susceptible to FUS/TLS or TDP-43 toxicity with a test agent; determining a level of mRNA processing in the population of neuronal cells or glial cells after the contacting step; and comparing the level of mRNA processing to a control; wherein a test agent that increases the level of mRNA processing relative to the control is identified as an agent useful in the treatment of ALS.

In various aspects, the present disclosure provides methods of identifying an agent useful in the treatment of ALS, comprising: contacting a first population of neuronal cells or glial cells that are suffering from or susceptible to FUS/TLS or TDP-43 toxicity with a test agent; determining a first number of viable cells in the first population after the contacting step; administering an NMD polypeptide to a second population of neuronal cells or glial cells that are suffering from or susceptible to FUS/TLS or TDP-43 toxicity; and determining a second number of viable cells in the second population after the administration step; wherein a first number of viable cells that is comparable to the second number of viable cells indicates the test agent is an agent useful in the treatment of ALS.

In various aspect, the present disclosure provides pharmaceutical compositions for treating ALS comprising an NMD polypeptide, a nucleic acid encoding an NMD polypeptide, or an activator of an NMD polypeptide, and a pharmaceutically acceptable excipient. In some embodiments, the pharmaceutical composition further comprising a targeting agent. In some embodiments, upon administration to a subject, the targeting agent selectively targets the composition to the brain.

In various aspect, the present disclosure provides methods of treating ALS in a human subject suffering from or susceptible to ALS, comprising: administering to the human subject a therapeutically effective amount of a UPF1 polypeptide, wherein the therapeutically effective amount is correlated with a statistically significant probability of reducing toxicity in a human neuronal cell or a human glial cell, thereby treating the ALS. In some embodiments, the subject has a mutation in an ALS2 gene, a VAPB gene, a SETX gene, a TDP-43 gene, a FUS/TLS gene, or an OPTN gene. In some embodiments, the subject does not have a mutation in a SOD1 gene.

### BRIEF DESCRIPTION OF THE DRAWINGS

The following figures are presented for the purpose of illustration only, and are not intended to be limiting.

FIG. 1A is a graphical representation of cell death of neurons following expression of UPF1. FIG. 1B is a graphical representation of cell death of neurons following expression of TDP-43 and UPF1.

All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Other features and advantages of the invention will be apparent from the following detailed description, and from the claims.

### DEFINITIONS

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Amelioration: As used herein, the term “amelioration” means the prevention, reduction or palliation of a state, or improvement of the state of a subject. Amelioration includes, but does not require, complete recovery or complete prevention of a disease condition.

Characteristic portion: As used herein, the term a “characteristic portion” of a substance, in the broadest sense, is one that shares some degree of sequence or structural identity with respect to the whole substance. In certain embodiments, a characteristic portion shares at least one functional characteristic with the intact substance. For example, in some embodiments, a “characteristic portion” of a polypeptide or protein is one that contains a continuous stretch of amino acids, or a collection of continuous stretches of amino acids, that together are characteristic of a polypeptide or protein. In some embodiments, each such continuous stretch generally contains at least 2, 5, 10, 15, 20, 50, or more amino acids. In some embodiments, such a continuous stretch includes certain residues whose position and identity are fixed; certain residues whose identity tolerates some variability (i.e., one of a few specified residues is accepted); and optionally certain residues whose identity is variable (i.e., any residue is accepted). In general, a characteristic portion of a substance (e.g., of a polypeptide or protein) is one that, in addition to the sequence and/or structural identity specified above, shares at least one functional characteristic with the relevant intact substance. In some embodiments, a characteristic portion may be biologically active.

Characteristic sequence: A “characteristic sequence” is a sequence that is found in all members of a family of polypeptides or nucleic acids, and therefore can be used by those of ordinary skill in the art to define members of the family.

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

Comparable: The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

Correlates: The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, or reduce: As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein.

NMD agent: As used herein, the term “NMD agent” refers to an NMD polypeptide, a nucleic acid that encodes an NMD polypeptide, or an agent that increases NMD polypeptide level and/or activity. In some embodiments, an NMD agent is a therapeutic agent.

NMD polypeptide: As used herein, the term “NMD polypeptide” refers to a polypeptide whose amino acid sequence includes at least one characteristic sequence of and/or shows at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71% or 70% identity with a protein involved in nonsense-mediated mRNA decay (e.g., UPF1, UPF2, UPF3, SMG1, SMG5, SMG6, or SMG7). A wide variety of NMD sequences from flies, vertebrates, and mammals are known in the art, such as those described herein; in some embodiments, an NMD polypeptide shares at least one characteristic sequence of and/or shows the specified degree of overall sequence identity with one of the UPF1, UPF2, UPF3, SMG1, SMG5, SMG6, or SMG7 set forth herein (each of which may be considered a “reference” NMD polypeptide). In some embodiments, an NMD polypeptide as described herein shares at least one biological activity with a reference NMD polypeptide as set forth herein. In some such embodiment, the shared biological activity relates to nonsense-mediated mRNA decay.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Providing: As used herein, the term “providing” refers to performing a manipulation that causes an entity of interest to be present at a level and/or with an activity higher than that observed under otherwise comparable conditions prior to or absent the manipulation. In some embodiments, providing consists of or comprises administering the entity itself (alone or as part of a composition); in some embodiment, providing consists of or comprises administering an agent that causes an increase in level and/or activity of the entity of interest. For example, where the entity of interest is or comprises a polypeptide, in some embodiments, “providing” the polypeptide consists of or comprises administering the polypeptide (e.g., to a cell, whether isolated or in an organism); in some embodiments, “providing” the polypeptide consists of or comprises administering a nucleic acid encoding the polypeptide; in some embodiments, “providing” the polypeptide consists of or comprises administering an agent that results in increased expression of an endogenous copy of the polypeptide (e.g., by stimulating one or more of transcription, RNA processing, translation, etc. and/or by inhibiting an inhibitor of one of these).

Reference: A “reference” entity, system, amount, set of conditions, etc., is one against which a test entity, system, amount, set of conditions, etc. is compared as described herein. For example, in some embodiments, a “reference” individual is a control individual who is not suffering from or susceptible to any form of ALS disease; in some embodiments, a “reference” individual is a control individual afflicted with the same form of ALS disease as an individual being treated, and optionally who is about the same age as the individual being treated (to ensure that the stages of the disease in the treated individual and the control individual(s) are comparable).

Subject: As used herein, the term “subject”, “individual”, or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.).

Target cell or target tissue: As used herein, the terms “target cell” or “target tissue” refers to any cell, tissue, or organism that is affected by ALS to be treated, or any cell, tissue, or organism in which a protein involved in ALS is expressed. In some embodiments, target cells, target tissues, or target organisms include those cells, tissues, or organisms in which there is a detectable or abnormally high amount of FUS or TDP-43 (e.g., comparable to that observed in patients suffering from or susceptible to ALS). In some embodiments, target cells, target tissues, or target organisms include those cells, tissues, or organisms that display a disease-associated pathology, symptom, or feature.

Therapeutic agent: As used herein, the phrase “therapeutic agent” refers to any agent that, when administered to a subject, has a therapeutic effect and/or elicits a desired biological and/or pharmacological effect.

Therapeutic regimen: As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease such as ALS. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g., transcription, processing, and/or translation of a particular gene product, such as a primary transcript or mRNA).

Therapeutically effective amount: As used herein, the term “therapeutically effective amount” refers to an amount of a therapeutic agent (e.g., an NMD polypeptide) which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the particular form of ALS being treated; the severity of the ALS; the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

Treatment: As used herein, the term “treatment” (also “treat” or “treating”) refers to any administration of a therapeutic agent (e.g., an NMD polypeptide) according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., ALS); in some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION

The present disclosure encompasses the surprising discovery that UPF1 can prevent neuronal toxicity due to TDP-43 or FUS/TLS. UPF1 is a protein involved in nonsense-mediated mRNA decay (NMD). Accordingly, the disclosure provides, among other things, various therapeutic modalities, including use of NMD polypeptides (e.g., UPF1, UPF2, UPF3, SMG1, SMG5, SMG6, or SMG7) to treat amyotrophic lateral sclerosis (ALS).

### Amyotrophic Lateral Sclerosis (ALS)

ALS, which exists as both inherited and random forms, is characterized by degeneration of spinal motor neurons, leading to paralysis and death. While most forms of ALS are sporadic and idiopathic (sALS), about 10% of cases are inherited in a Mendelian fashion and are designated familial ALS (fALS). The present invention provides compositions and methods useful in treating ALS.

Using genetic analysis, several genes that cause fALS have been identified. The first mutations were identified in SOD1, which encodes the ubiquitously expressed copper/zinc superoxide dismutase. These variants are involved in about 20% of fALS cases worldwide (Rosen et al., Nature 362:59-62 (1993)). Other genes involved in fALS include genes coding for alsin (ALS2), vesicle associated membrane protein B (VAPB) (Nishimura et al., Am. J. Hum. Genet. 75:822-831 (2004)), senataxin (SETX) (Chen et al., Am. J. Hum. Genet. 74:1128-1135 (2004)), TAR-DNA-binding protein (TDP-43) (Sreedharan et al., Science 319:1668-1672 (2008)), fused in sarcoma or translocated in liposarcoma (FUS/TLS) (Kwiatkowski et al., Science 323:1205-1208 (2009); Vance et al., Science 323:1208-1211 (2009)), and optineurin (OPTN) (Maruyama et al., Nature 465:223-226 (2010)). FUS/TLS is a nucleic acid binding protein that, when mutated, can cause a subset of fALS and can also increase risk for the sporadic disease. Although FUS/TLS is normally located predominantly in the nucleus, pathogenic mutant forms of FUS/TLS traffic to, and form inclusions in, the cytoplasm of affected spinal motor neurons or glia.

Studies of these genes have provided insight into the biochemical processes that may underlie ALS. Putative mechanisms of toxicity targeting motor neurons include glutamate excitotoxicity, oxidative damage, proteasome inhibition, mitochondrial dysfunction, ER stress, axonal transport defects, growth factor signaling deficiency, and glial cell dysfunction (Rothstein et al., Ann. Neurol. 65:S3-S9 (2009); Ilieva et al., J. Cell Biol. 187:761-772 (2009)).

### Nonsense-Mediated mRNA Decay

In mammalian cells, expression of protein-encoding genes requires a series of steps in which pre-mRNA is processed to mRNA in the nucleus before mRNA is translated into protein in the cytoplasm. These steps are subject to quality control to ensure that only completely processed mRNA is exported to the cytoplasm (see, e.g., Maquat et al., Cell 104:173-176 (2001)). One form of quality control, called mRNA surveillance or nonsense-mediated mRNA decay (NMD), degrades mRNAs that prematurely terminate translation more than 50-55 nucleotides upstream of an exon-exon junction as a means to prevent the synthesis of potentially harmful truncated proteins (sec, e.g., Maquat, J. Cell Sci. 118:1773-1776 (2005); Nicholson et al., Biochem. Soc. Trans. 38:1615-20 (2010)). A number of proteins are involved in NMD in mammalian cells, including UPF1, UPF2, UPF3, SMG1, SMG5, SMG6, and SMG7 (Wittkopp et al., Mol. Cell. Biol. 29:3517-3528 (2009); Rehwinkel et al, Trends Biochem. Sci. 31:639-646 (2006); Rehwinkel et al., RNA 11:1530-1544 (2005)). According to the present disclosure, any NMD polypeptides can be used to treat ALS in methods described herein.

Nucleic Acid Sequences Encoding NMD Polypeptides

Methods and compositions described herein include, for example, nucleic acids encoding NMD polypeptides (e.g., UPF1, UPF2, UPF3, SMG1, SMG5, SMG6, or SMG7). According to the present disclosure, such nucleic acids (and polypeptides) are useful in the treatment of ALS. In some embodiments, such nucleic acids have or include nucleotide sequences as set forth in SEQ ID NO:1, 3, 5, 7, 9, 11, or 13, or characteristic sequence elements thereof or therein. In some embodiments, useful nucleic acids show at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% overall sequence identity with one or more of SEQ ID NO:1, 3, 5, 7, 9, 11, or 13. Alternatively or additionally, in some embodiments, useful nucleic acids include at least 5, 6, 7, 8, 9, 10, 11, 12, 13, 14, 15, 16, 17, 18, 19, 20, 21, 22, 23, 24, 25 or more contiguous residues found in SEQ ID NO:1, 3, 5, 7, 9, 11 or 13. In some embodiments, useful nucleic acids are generated in vitro; in some embodiments, useful nucleic acids are generated in vivo. In some embodiments, useful nucleic acids are generated using genetic engineering techniques (e.g., for production and/or mutagenesis of a reference sequence). To give but a few examples, in some embodiments, nucleic acid variants (e.g., of SEQ ID NO:1, 3, 5, 7, 9, 11 or 13) are generated using techniques such as site directed mutagenesis, random chemical mutagenesis, Exonuclease III deletion procedures, and standard cloning techniques. In some embodiments, useful nucleic acids are generating using chemical synthesis and/or modification procedures.

A variety of methods of making nucleic acids that are “variants” with respect to a reference nucleic acid (e.g., a naturally-occurring or other reference nucleic acid) are well known in the art. These include, for example, procedures in which nucleic acid sequences obtained from natural isolates are modified to generate nucleic acids that encode polypeptides having characteristics that enhance their value in industrial or laboratory applications. In such some embodiments of such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be created using error prone PCR (see, e.g., Leung et al., Technique 1:11-15, 1989; and Caldwell et al., PCR Methods Applic. 2:28-33, 1992). In error prone PCR, PCR is performed under conditions where the copying fidelity of the DNA polymerase is low, such that a high rate of point mutations is obtained along the entire length of the PCR product. Briefly, in such procedures, nucleic acids to be mutagenized are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized, 30 pmole of each PCR primer, a reaction buffer comprising 50 mM KCl, 10 mM Tris HCl (pH 8.3), and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min, 45° C. for 1 min, and 72° C. for 1 min. However, it will be appreciated that these parameters can be varied as appropriate. The mutagenized nucleic acids are then cloned into an appropriate vector and the activities of the polypeptides encoded by the mutagenized nucleic acids are evaluated.

Variants can also be created using oligonucleotide directed mutagenesis to generate site-specific mutations in any cloned DNA of interest. Oligonucleotide mutagenesis is described in, for example, Reidhaar-Olson et al., Science 241:53-57 (1988). Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408. Still another method of generating variants is sexual PCR mutagenesis. In sexual PCR mutagenesis, forced homologous recombination occurs between DNA molecules of different, but highly related, DNA sequence in vitro as a result of random fragmentation of the DNA molecule based on sequence homology. This is followed by fixation of the crossover by primer extension in a PCR reaction. Sexual PCR mutagenesis is described in, for example, Stemmer, Proc. Natl. Acad. Sci., USA 91:10747-10751 (1994).

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence in one of these strains will generate random mutations within the DNA. Mutator strains suitable for use for in vivo mutagenesis are described in, for example, PCT Publication No. WO 91/16427.

Variants can also be generated using cassette mutagenesis. In cassette mutagenesis, a small region of a double stranded DNA molecule is replaced with a synthetic oligonucleotide “cassette” that differs from the native sequence. The oligonucleotide often contains a completely and/or partially randomized native sequence. Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described in, for example, Arkin et al., Proc. Natl. Acad. Sci., USA 89:7811-7815 (1992).

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described in, for example, Delegrave et al., Biotech. Res. 11:1548-1552 (1993). Random and site-directed mutagenesis are described in, for example, Arnold, Curr. Opin. Biotech. 4:450-455 (1993). In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

In some embodiments, nucleic acids for use in accordance with the present disclosure comprise naturally-occurring nucleotide residues. In some embodiments, nucleic acids for use in accordance with the present disclosure include one or more nucleotide “analogs”. A nucleotide analog is a nucleotide (i.e., an entity that is incorporated into a nucleic acid polymer without significantly disrupting the structure and/or function of that polymer) whose chemical structure differs from that of reference naturally-occurring ribonucleic or deoxyribonucleic acid residues adenine, guanine, cytosine, thymine, and uracil. In some embodiments, a nucleotide analog differs from its reference nucleotide at the base moiety, sugar moiety, and/or phosphate backbone. In some embodiments, a nucleotide analog contributes to one or more altered features in a nucleic acid polymer into which it is incorporated as compared with a comparable nucleic acid polymer containing its reference nucleotide rather than the analog. For example, in some embodiments, such analog-containing polymer shows improved, stability, hybridization, and/or solubility.

In some embodiments, base moiety alterations found in nucleotide analogs include deoxyuridine for deoxythymidine and 5-methyl-2′-deoxycytidine or 5-bromo-2′-deoxycytidine for deoxycytidine. In some embodiments, sugar moiety alterations found in nucleotide analogs include modification of the 2′ hydroxyl of the ribose sugar to form 2′-O-methyl or 2′-O-allyl sugars. In some embodiments, deoxyribose phosphate backbone alterations found in nucleotide analogs include morpholino nucleic acids, in which each base moiety is linked to a six-membered, morpholino ring, or peptide nucleic acids, in which the deoxyphosphate backbone is replaced by a pseudopeptide backbone and the four bases are retained (see, e.g., Summerton et al., Antisense Nucleic Acid Drug Dev. 7:187-195 (1997); Hyrup et al., Bioorgan. Med. Chem. 4:5-23 (1996)). Alternatively or additionally, nucleotide analogs may have a phosphorothioate or phosphorodithioate backbone, a phosphoroamidite, or an alkyl phosphotriester backbone.

In certain instances, an NMD polynucleotide or variant for use in accordance with the present disclosure includes alterations to codon(s) to optimize for expression in a particular host cell. For example, for expression in E. coli, an NMP polynucleotide or variant can include one or more altered codons as described in, e.g., Grosjean et al., Gene 18:199-209 (1982).

### NMD Polypeptides

In some embodiments, methods and compositions described utilize NMD polypeptides (e.g., UPF1, UPF2, UPF3, SMG1, SMG5, SMG6, or SMG7 polypeptides). According to the present disclosure, such polypeptides are useful in the treatment of ALS. In some embodiments, such polypeptides useful in the practice of the present disclosure have or include amino acid sequences as set forth in SEQ ID NO:2, 4, 6, 8, 10, 12, or 14, or characteristic sequence elements thereof or therein. In some embodiments, useful polypeptides show at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% overall sequence identity with one or more of SEQ ID NO:2, 4, 6, 8, 10, 12, or 14. Alternatively or additionally, in some embodiments, useful polypeptides include at least about 5, 10, 15, 20, 25, 30, 35, 40, 45, 50, 75, 100, or 150 or more contiguous amino acid residues found in SEQ ID NO: 2, 4, 6, 8, 10, 12, or 14.

In some embodiments, a useful polypeptide differs from its reference polypeptide (e.g., a polypeptide having or including an amino acid sequence as set forth in SEQ ID NO:2, 4, 6, 8, 10, 12, or 14, or characteristic sequence elements thereof or therein) by one or more amino acid residues. For example, in some embodiments, the difference is a conservative or nonconservative substitution of one or more amino acid residues. Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

In some embodiments, useful NMD polypeptides include a substituent group on one or more amino acid residues. Still other useful polypeptides are associated with (e.g., fused, linked, or coupled to) another moiety (e.g., a peptide or molecule). For example, useful NMD polypeptides can be fused, linked, or coupled to an amino acid sequence (e.g., a leader sequence, a secretory sequence, a proprotein sequence, a second polypeptide, or a sequence that facilitates purification, enrichment, or stabilization of the polypeptide). In certain other embodiments, a polypeptide includes a targeting agent, e.g., a targeting agent described herein.

A variety of methods of making polypeptides are known in the art and can be used to make NMD polypeptides. For example, NMD polypeptides can be recombinantly produced by utilizing a host cell system engineered to express a nucleic acid encoding an NMD polypeptide (e.g., a nucleic acid described herein). Alternatively or additionally, an NMD polypeptide can be produced by activating an endogenous gene (e.g., a nucleic acid encoding an NMD polypeptide present endogenously in a cell). Alternatively or additionally, an NMD polypeptide can be partially or fully prepared by chemical synthesis. Alternatively or additionally, an NMD polypeptide can be purified from natural sources.

Where an NMD polypeptide is recombinantly produced, any expression system can be used. Known expression systems include, without limitation, for example, egg, baculovirus, plant, yeast, or mammalian cells.

In some embodiments, an NMD polypeptide suitable for use in methods described herein are produced in mammalian cells. Non-limiting examples of mammalian cells that can be used include BALB/c mouse myeloma line (NSO/1, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59,1977); human fibrosarcoma cell line (e.g., HT1080); baby hamster kidney cells (BHK, ATCC CCL 10); Chinese hamster ovary cells+/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

### Targeting Agents

An NMD agent described herein can be provided in association with and/or can include a targeting agent.

The present disclosure is not limited to any particular targeting agent, and a variety of targeting agents can be used. Examples of such targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to target cells or target tissues (e.g., receptors on target cells or target tissues).

Targeting agents can be associated with NMD agents in any of a number of ways. For example, polypeptide targeting agents can be coupled to or fused to an NMD polypeptide. In other embodiments, a targeting agent is associated (e.g., covalently or noncovalently bound) to an NMD agent with either short (e.g., direct coupling), medium (e.g., using small-molecule bifunctional linkers such as SPDP (Pierce Biotechnology, Inc., Rockford, Ill.)), or long (e.g., PEG bifunctional linkers (Nektar Therapeutics, Inc., San Carlos, Calif.)) linkages.

In some instances, targeting agents are or comprise antigen binding proteins or antibodies or binding portions thereof. Antibodies can be generated to allow for specific targeting of antigens or immunogens (e.g., target cell or target tissue specific antigens). Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab′, Fab, F(ab′)₂); or biosynthetic antibodies, e.g., single chain antibodies, single domain antibodies (DAB), Fvs, or single chain Fvs (scFv) (see, e.g., in Harlow et al., Using Antibodies: A Laboratory Manual: Portable Protocol I. Cold Spring Harbor Laboratory (Dec. 1, 1998); Zola, Monoclonal Antibodies: Preparation and Use of Monoclonal Antibodies and Engineered Antibody Derivatives, Springer Verlag (Dec. 15, 2000; 1st edition)). Antibody attachment can be performed by any known method e.g., through standard covalent binding to free amine groups (see, e.g., Torchilin et al., Hybridoma 6:229-240 (1987); Torchilin et al, Biochim. Biophys. Acta 1511:397-411 (2001); Masuko et al., Biomacromol. 6:800-884 (2005)).

In some instances, a targeting agent is or comprises a nucleic acid (e.g., RNA or DNA). In some examples, nucleic acid targeting agents are designed to hybridize by base pairing to a particular nucleic acid (e.g., chromosomal DNA, mRNA, or ribosomal RNA). In some situations, nucleic acid targeting agents bind a ligand on a target cell or target tissue. For example, a nucleic acid can bind human nerve growth factor (Binkley et al., Nuc. Acids Res. 23:3198-205 (1995)). Nucleic acids that bind ligands can be identified by known methods, such as SELEX procedures (see, e.g., U.S. Pat. Nos. 5,475,096; 5,270,163; and 5,475,096; and WO 97/38134; WO 98/33941; and WO 99/07724). In some embodiments, targeting agents can be or comprise aptamers, for example that bind to particular sequences.

In some embodiments, a targeting agent binds to a receptor on the surface of a brain cell to facilitate cellular uptake. For example, a targeting agent can be mannose-6-phosphate (M6P), bis-phosphorylated oligosaccharides, or IGF-II, which are useful for targeting the cation-independent mannose-6-phosphate receptor (CI-MPR) on a brain cell. In some embodiments, a targeting agent is or comprises ascorbate, which is taken up by a sodium-dependent-vitamin C transporter (SVCT2), (see, e.g., Tsukaguchi et al., Nature 399:70-75 (1999)), which is useful for targeting to a brain cell.

### Therapeutic Administration

NMD agents (e.g., NMD polynucleotides, a nucleic acid encoding an NMD polypeptide, or an agent that increases NMD polypeptide level and/or activity) described herein can be used to treat ALS, e.g., subjects suffering from or susceptible to ALS. The route and/or mode of administration of an NMD agent described herein can vary depending upon the desired results. One with skill in the art, i.e., a physician, is aware that dosage regimens can be adjusted to provide the desired response, e.g., a therapeutic response.

Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. The mode of administration is left to the discretion of the practitioner.

In some instances, an NMD agent described herein (e.g., a pharmaceutical formulation of an NMD agent) can effectively cross the blood brain barrier and enter the brain. In other instances, an NMD agent can be delivered using techniques designed to permit or to enhance the ability of the formulation to cross the blood-brain barrier. Such techniques are known in the art (e.g., WO 89/10134; Cloughesy et al., J. Neurooncol. 26:125-132 (1995); and Begley, J. Pharm. Pharmacol. 48:136-146 (1996)). Components of a formulation can also be modified (e.g., chemically) using methods known in the art to facilitate their entry into the CNS.

For example, physical methods of transporting compositions across the blood-brain barrier include, but are not limited to, circumventing the blood-brain barrier entirely, or by creating openings in the blood-brain barrier. Circumvention methods include, but are not limited to, direct injection into the brain (see e.g., Papanastassiou et al., Gene Therapy 9:398-406 (2002)) and implanting a delivery device in the brain (see e.g., Gill et al., Nature Med. 9:589-595 (2003); and Gliadel Wafers™, Guildford Pharmaceutical). Methods of creating openings in the barrier include, but are not limited to, ultrasound (see e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding an NMD agent (see, e.g., U.S. Patent Publ. No. 20030083299).

Lipid-based methods can also be used to transport an NMD agent across the blood-brain barrier. Exemplary, nonlimiting methods include encapsulating an NMD agent in liposomes that are coupled to a targeting agent described herein (e.g., an antibody that binds to receptors on vascular endothelium of the blood-brain barrier (see, e.g., U.S. Patent Publ. No. 20020025313). In certain other embodiments, a targeting agent is coated in low-density lipoprotein particles (see, e.g., U.S. Patent Publ. No. 20040204354) or apolipoprotein E (see, e.g., U.S. Patent Publ. No. 20040131692).

In some embodiments, an NMD agent is delivered to the CNS of a subject, e.g., by administering into the cerebrospinal fluid (CSF) of a subject in need of treatment. As used herein, intrathecal administration (also referred to as intrathecal injection) refers to an injection into the spinal canal (intrathecal space surrounding the spinal cord). Various techniques may be used including, without limitation, lateral cerebroventricular injection through a burrhole or cisternal or lumbar puncture or the like. Exemplary methods are described in Lazorthes et al., Adv. Tech. Stand. Neurosurg. 18:143-192 (1991), and Omaya, Cancer Drug Deliv. 1:169-179 (1984).

In some instances, an NMD agent described herein is administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers. In some situations, an NMD agent described herein is introduced into the central nervous system, circulatory system or gastrointestinal tract by any suitable route, including intraventricular injection, intrathecal injection, paraspinal injection, epidural injection, enema, and by injection adjacent to a peripheral nerve.

Specifically, various devices can be used for intrathecal delivery of NMD agents described herein. In some embodiments, a device for intrathecal administration contains a fluid access port (e.g., injectable port); a hollow body (e.g., catheter) having a first flow orifice in fluid communication with the fluid access port and a second flow orifice configured for insertion into spinal cord; and a securing mechanism for securing the insertion of the hollow body in the spinal cord. Various other devices may be used to effect intrathecal administration of a therapeutic composition. For example, formulations containing NMD agents can be administered using an Ommaya reservoir that is in common use for intrathecally administering drugs for meningeal carcinomatosis (Lancet 2:983-84, 1963). More specifically, in this method, a ventricular tube is inserted through a hole formed in the anterior horn and is connected to an Ommaya reservoir installed under the scalp, and the reservoir is subcutaneously punctured to intrathecally deliver an NMD agent, which is injected into the reservoir. Other devices for intrathecal administration of therapeutic compositions or formulations to an individual are described in U.S. Pat. No. 6,217,552. Alternatively, an NMD agent can be intrathecally given, for example, by a single injection, or continuous infusion. It should be understood that the dosage treatment may be in the form of a single dose administration or multiple doses.

In some embodiments, intrathecal administration can be performed by either lumbar puncture (i.e., slow bolus) or via a port-catheter delivery system (i.e., infusion or bolus).

Relative to intravenous administration, a single dose volume suitable for intrathecal administration is typically small. Typically, intrathecal delivery maintains the balance of the composition of the CSF as well as the intracranial pressure of the subject. In some embodiments, intrathecal delivery is performed absent the corresponding removal of CSF from a subject. In some embodiments, a suitable single dose volume may be e.g., less than about 10 ml, 8 ml, 6 ml, 5 ml, 4 ml, 3 ml, 2 ml, 1.5 ml, 1 ml, or 0.5 ml. In some embodiments, a suitable single dose volume may be about 0.5-5 ml, 0.5-4 ml, 0.5-3 ml, 0.5-2 ml, 0.5-1 ml, 1-3 ml, 1-5 ml, 1.5-3 ml, 1-4 ml, or 0.5-1.5 ml. In some embodiments, intrathecal delivery according to the present invention involves a step of removing a desired amount of CSF first. In some embodiments, less than about 10 ml (e.g., less than about 9 ml, 8 ml, 7 ml, 6 ml, 5 ml, 4 ml, 3 ml, 2 ml, 1 ml) of CSF is first removed before intrathecal administration. In those cases, a suitable single dose volume may be e.g., more than about 3 ml, 4 ml, 5 ml, 6 ml, 7 ml, 8 ml, 9 ml, 10 ml, 15 ml, or 20 ml.

Pulmonary administration can also be employed, e.g., by use of an inhaler or nebulizer, and formulation with an aerosolizing agent, or via perfusion in a fluorocarbon or synthetic pulmonary surfactant.

An NMD agent described herein can be formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one situation, the physiologically acceptable excipients are sterile when administered to an animal. The physiologically acceptable excipient should be stable under the conditions of manufacture and storage and should be preserved against the contaminating action of microorganisms. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. An NMD agent described herein can be suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

In other instances, an NMD agent described herein is formulated for intravenous administration. Compositions for intravenous administration can comprise a sterile isotonic aqueous buffer. The compositions can also include a solubilizing agent. Compositions for intravenous administration can optionally include a local anesthetic such as lidocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where an NMD agent described herein is administered by infusion, it can be dispensed, for example, with an infusion bottle containing sterile pharmaceutical grade water or saline. Where an NMD agent described herein is administered by injection, an ampule of sterile water for injection or saline can be provided so that the ingredients can be mixed prior to administration.

An NMD agent described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of an NMD agent described herein that is effective for treating ALS can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner.

Compositions described herein (e.g., therapeutically effective amounts of compositions described herein) can be administered as single administrations or as multiple administrations. Such compositions can be administered at regular intervals, depending on the nature, severity and extent of the subject's condition (e.g., ALS). In some embodiments, a therapeutically effective amount of a therapeutic agent (e.g., an NMD agent) is administered intrathecally periodically at regular intervals (e.g., once every year, once every six months, once every five months, once every three months, bimonthly (once every two months), monthly (once every month), biweekly (once every two weeks), or weekly).

As used herein, the term “therapeutically effective amount” is largely determined based on the total amount of the therapeutic agent contained in pharmaceutical compositions described herein. Generally, a therapeutically effective amount is sufficient to achieve a meaningful benefit to a subject (e.g., treating, modulating, curing, preventing and/or ameliorating ALS). For example, a therapeutically effective amount can be an amount sufficient to achieve a desired therapeutic and/or prophylactic effect, such as an amount sufficient to treat ALS or the symptoms thereof. Generally, the amount of a therapeutic agent (e.g., an NMD agent) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays can optionally be employed to identify optimal dosage ranges. A therapeutically effective amount can be administered in a dosing regimen that can include multiple unit doses.

In some embodiments, a therapeutically effective dose ranges from about 0.005 mg/kg brain weight to 500 mg/kg brain weight, e.g., from about 0.005 mg/kg brain weight to 400 mg/kg brain weight, from about 0.005 mg/kg brain weight to 300 mg/kg brain weight, from about 0.005 mg/kg brain weight to 200 mg/kg brain weight, from about 0.005 mg/kg brain weight to 100 mg/kg brain weight, from about 0.005 mg/kg brain weight to 90 mg/kg brain weight, from about 0.005 mg/kg brain weight to 80 mg/kg brain weight, from about 0.005 mg/kg brain weight to 70 mg/kg brain weight, from about 0.005 mg/kg brain weight to 60 mg/kg brain weight, from about 0.005 mg/kg brain weight to 50 mg/kg brain weight, from about 0.005 mg/kg brain weight to 40 mg/kg brain weight, from about 0.005 mg/kg brain weight to 30 mg/kg brain weight, from about 0.005 mg/kg brain weight to 25 mg/kg brain weight, from about 0.005 mg/kg brain weight to 20 mg/kg brain weight, from about 0.005 mg/kg brain weight to 15 mg/kg brain weight, from about 0.005 mg/kg brain weight to 10 mg/kg brain weight.

In some embodiments, a therapeutically effective dose is greater than about 0.1 mg/kg brain weight, greater than about 0.5 mg/kg brain weight, greater than about 1.0 mg/kg brain weight, greater than about 3 mg/kg brain weight, greater than about 5 mg/kg brain weight, greater than about 10 mg/kg brain weight, greater than about 15 mg/kg brain weight, greater than about 20 mg/kg brain weight, greater than about 30 mg/kg brain weight, greater than about 40 mg/kg brain weight, greater than about 50 mg/kg brain weight, greater than about 60 mg/kg brain weight, greater than about 70 mg/kg brain weight, greater than about 80 mg/kg brain weight, greater than about 90 mg/kg brain weight, greater than about 100 mg/kg brain weight, greater than about 150 mg/kg brain weight, greater than about 200 mg/kg brain weight, greater than about 250 mg/kg brain weight, greater than about 300 mg/kg brain weight, greater than about 350 mg/kg brain weight, greater than about 400 mg/kg brain weight, greater than about 450 mg/kg brain weight, greater than about 500 mg/kg brain weight.

In some embodiments, a therapeutically effective dose can be expressed as mg/kg body weight. As one skilled in the art would appreciate, brain weights and body weights can be correlated (see, e.g., Dekaban, Ann. Neurol. 4:345-56 (1978)).

In some embodiments, a therapeutically effective dose can be expressed as mg/15 cc of CSF. As one skilled in the art would appreciate, therapeutically effective doses based on brain weights and body weights can be converted to mg/15 cc of CSF. For example, the volume of CSF in adult humans is approximately 150 mL (Johanson et al., Cerebrospinal Fluid Res. 14:5:10 (2008)). Therefore, single dose injections of 0.1 mg to 50 mg protein to adults would be approximately 0.01 mg/15 cc of CSF (0.1 mg) to 5.0 mg/15 cc of CSF (50 mg) doses in adults.

It is to be further understood that for any particular subject, specific dosage regimens can be adjusted over time according to the individual need and the professional judgment of the person administering or supervising the administration of an NMD agent and that dosage ranges set forth herein are exemplary only and are not intended to limit the scope or practice of the claimed invention.

In some instances, a pharmaceutical composition described herein is in unit dosage form, e.g., as a tablet, capsule, powder, solution, suspension, emulsion, granule, or suppository. In such form, the pharmaceutical composition can be sub-divided into unit doses containing appropriate quantities of an NMD agent described herein. The unit dosage form can be a packaged pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

### Gene Therapy

In embodiments in which an NMD agent consists of or comprises a nucleic acid encoding an NMD polypeptide, the present disclosure includes methods of administering such nucleic acid to a subject to treat ALS.

In some embodiments, a nucleic acid encoding an NMD polypeptide is inserted into a viral vector for delivery to a subject. For example, retrovirus vectors can be used as a recombinant delivery system for transferring nucleic acids encoding NMD polypeptides vivo (see, e.g., Dropulic, Hum. Gene Ther. 22:649-57 (2011); and Kumar et al., Curr. Gene Ther. 11:144-53 (2011)). Retroviruses useful in methods of the present disclosure include, but are not limited to, murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), FBR murine osteosarcoma virus (FBR MSV), Moloney murine sarcoma virus (Mo-MSV), Abelson murine leukemia virus (A-MLV), Avian myelocytomatosis virus-29 (MC29), Avian erythroblastosis virus (AEV) and all other retroviridiae including lentiviruses (see, e.g., Coffin et al., “Retroviruses”, 1997 Cold Spring Harbor Laboratory Press Eds: J M Coffin, S M Hughes, H E Varmus, pp 758-763)). A replication defective retrovirus can be packaged into virions that can be used to infect a target cell through the use of a helper virus by standard techniques (see, e.g., Current Protocols in Molecular Biology, Ausubel, F. M. et al. (eds.) Greene Publishing Associates, (1989), Sections 9.10-9.14).

In other embodiments, adenovirus-derived vectors are used to deliver nucleic acids encoding NMD polypeptides. The genome of an adenovirus can be manipulated such that it encodes and expresses an NMD polypeptide, but is inactivated in terms of its ability to replicate in a normal lytic viral life cycle (see, e.g., Berkner et al. (1988) BioTechniques 6:616;Rosenfeld et al. (1991) Science 252:431-434; and Rosenfeld et al. (1992) Cell 68:143-155). Suitable adenoviral vectors useful in the methods of the present disclosure include those derived from the adenovirus strain Ad type 5 d1324 or other strains of adenovirus (e.g., Ad2, Ad3, Ad7 etc.).

In some embodiments, an adeno-associated virus (AAV) is used to deliver a nucleic acid encoding an NMD polypeptide (see, e.g., Muzyczka et al. (1992) Curr. Topics in Micro. and Immunol. 158:97-129). A variety of nucleic acids have been introduced into different cell types using AAV vectors (see, e.g., Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Particularly useful AAVs include those that normally infect humans (e.g., serotypes 1, 2, 3A, 3B, 4, 5, and 6) or primates (e.g., serotypes 1 and 4).

In other embodiments, non-viral methods are useful to deliver a nucleic acid encoding an NMD polypeptide to a subject. Such nonviral methods of gene transfer can exploit mechanisms normally used by mammalian cells for uptake and intracellular transport of macromolecules. For example, liposomal delivery systems, poly-lysine conjugates, and artificial viral envelopes can be used. In some embodiments, a nucleic acid encoding an NMD polypeptide is entrapped in liposomes bearing positive charges on their surface (e.g., lipofectins). In some embodiments, a liposome can be conjugated to a targeting agent described herein (see, e.g., Mizuno et al. (1992) No Shinkei Geka 20:547-551).

Certain cationic polymers (“complexation agents”) known to spontaneously bind to and condense nucleic acids into nanoparticles can also be used including, e.g., naturally occurring proteins, peptides, or derivatives, as well as synthetic cationic polymers such as polyethylenimine (PEI), polylysine (PLL), etc. Many useful polymers contain both chargeable amino groups, to allow for ionic interaction with negatively charged DNA phosphate, and a degradable region, such as a hydrolyzable ester linkage. Examples of these include, without limitation, poly (alpha-(4-aminobutyl)-L-glycolic acid), network poly(amino ester), and poly (beta-amino esters). Such complexation agents can protect DNA against degradation, e.g., by nucleases, serum components, etc., and create a less negative surface charge, which may facilitate passage through hydrophobic membranes (e.g., cytoplasmic, lysosomal, endosomal, nuclear) of the cell. Certain complexation agents facilitate intracellular trafficking events such as endosomal escape, cytoplasmic transport, and nuclear entry, and can dissociate from the nucleic acid.

### Cell-Based Therapy

An NMD polynucleotide can also be advantageously provided to a cell ex vivo, followed by administration of the living cell to the subject. In some embodiments, primary or secondary cells are genetically engineered to express an NMD polypeptide. Such cells can be obtained from a variety of tissues and include cell types which can be maintained propagated in culture. For example, primary and secondary cells include fibroblasts, endothelial cells, glial cells, and neural cells. In some embodiments, primary cells are obtained from an individual to whom a genetically engineered primary or secondary cells is to be administered. Primary cells can also be obtained from a donor (other than the recipient) of the same species or another species (e.g., mouse, rat, rabbit, cat, dog, pig, cow, bird, sheep, goat, horse).

Primary or secondary cells (e.g., of vertebrate or mammalian origin) can be transfected with a nucleic acid encoding an NMD polypeptide. In some embodiments, a cell is transfected with an exogenous nucleic acid sequence that includes a nucleic acid encoding an NMD polypeptide and an additional nucleic acid sequence (e.g., a regulatory sequence, e.g., a promoter, which causes expression, e.g., inducible expression or upregulation, of an endogenous NMD sequence). Transfected primary or secondary cells may also include DNA encoding a selectable marker which confers a selectable phenotype upon them, facilitating their identification and isolation.

Methods for treating disease by implanting a cell that has been modified to express a recombinant protein are also well known. See, for example, U.S. Pat. No. 5,399,346, disclosing methods for introducing a nucleic acid into a primary human cell for introduction into a human. Although use of human cells for ex vivo therapy is preferred in some embodiments, other cells such as bacterial cells may be implanted in a subject's vasculature, continuously releasing a therapeutic agent. See, for example, U.S. Pat. Nos. 4,309,776 and 5,704,910.

### Kits

An NMD agent described herein (e.g., a pharmaceutical composition comprising an NMD agent) can be provided in a kit. In some instances, the kit includes (a) a container that contains an NMD agent described herein (e.g., a pharmaceutical composition comprising an NMD agent) and, optionally (b) informational material. The informational material can be descriptive, instructional, marketing or other material that relates to the methods described herein and/or the use of an NMD agent, e.g., for therapeutic benefit.

The informational material of the kits is not limited in its form. In some instances, the informational material can include information about production of an NMD agent, molecular weight of an NMD agent, concentration, date of expiration, batch or production site information, and so forth. In other situations, the informational material relates to methods of administering an NMD agent, e.g., in a suitable amount, manner, or mode of administration (e.g., a dose, dosage form, or mode of administration described herein). The method can be a method of treating a subject having ALS.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about an NMD agent therein and/or their use in the methods described herein. The informational material can also be provided in any combination of formats.

In addition to an NMD agent, the kit can include other ingredients, such as a solvent or buffer, a stabilizer, or a preservative. The kit can also include other agents, e.g., a second or third agent, e.g., other therapeutic agents. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for an NMD agent or other agents. In some cases, the kit contains separate containers, dividers or compartments for an NMD agent and informational material. For example, an NMD agent can be contained in a bottle, vial, or syringe, and the informational material can be contained in a plastic sleeve or packet. In other situations, the separate elements of the kit are contained within a single, undivided container. For example, an NMD agent can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of an NMD agent. The containers can include a unit dosage, e.g., a unit that includes an NMD agent. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of an NMD agent, e.g., a syringe or other suitable delivery device. The device can be provided preloaded with an NMD agent, e.g., in a unit dose, or can be empty, but suitable for loading.

### Treatment of ALS

The present invention encompasses the surprising finding that NMD agents are useful, among other things, in the treatment or prevention (i.e., delay of onset) of ALS. UPF1 was initially identified as one of many genes able to rescue toxicity mediated by FUS/TLS in a yeast model (Ju et al., PLoS Biol. 9:e1001052 (2011)). However, the present finding that expressing UPF1 in neuronal cells expressing FUS/TLS or TDP-43 reduces cellular toxicity is surprising, especially given the finding that expression of UPF1 had no effect on the cytoplasmic levels of FUS/TLS or TDP-43 in the neuronal cells. Accordingly, in some embodiments, an NMD agent is provided to the central nervous system of a subject, e.g., a subject suffering from or susceptible to ALS. In some embodiments, an NMD agent is provided to one or more of target cells or tissues of brain, spinal cord, and/or peripheral organs. In some embodiments, target cells or tissues include those cells or tissues that display a disease-associated pathology, symptom, or feature. In some embodiments, target cells or tissues include those cells or tissues in which TDP-43 or FUS/TLS is expressed at an elevated level, e.g., cells in which TDP-43 or FUS/TLS is expressed at an elevated level in the cytoplasm of the cells. As used herein, a target tissue may be a brain target tissue, a spinal cord target tissue and/or a peripheral target tissue.

Compositions described herein can be provided directly into the CNS of a subject suffering from or at risk of developing ALS, thereby achieving a therapeutic concentration within the affected cells and tissues of the CNS (e.g., the brain). For example, one or more NMD agents can be provided to target cells or tissues of the brain, spinal cord and/or peripheral organs to treat ALS. As used herein, the term “treat” or “treatment” refers to amelioration of one or more symptoms associated with the disease, prevention or delay of the onset of one or more symptoms of the disease, and/or lessening of the severity or frequency of one or more symptoms of the disease.

In some embodiments, treatment refers to partially or complete alleviation, amelioration, relief, inhibition, delaying onset, reducing severity and/or incidence of neurological impairment in a patient suffering from or susceptible to ALS. As used herein, the term “neurological impairment” includes various symptoms associated with impairment of the central nervous system (e.g., the brain and spinal cord). Symptoms of neurological impairment may include, for example, developmental delay, progressive cognitive impairment, hearing loss, impaired speech development, deficits in motor skills, hyperactivity, aggressiveness and/or sleep disturbances, among others.

In some embodiments, treatment refers to decreased toxicity of various cells or tissues. In some embodiments, treatment refers to decreased neuronal toxicity due to FUS or TDP-43 in brain target tissues, spinal cord neurons, and/or peripheral target tissues. In certain embodiments, toxicity is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. In some embodiments, toxicity is decreased by at least 1-fold, 2-fold, 3-fold, 4-fold, 5-fold, 6-fold, 7-fold, 8-fold, 9-fold or 10-fold as compared to a control. In some embodiments, toxicity is measured by tests known to those of ordinary skill in the art including, but not limited to, neuroimaging methods (e.g., CT scans, MRI, functional MRI, etc.).

In certain embodiments, treatment according to the present disclosure results in a reduction (e.g., about a 5%, 10%, 15%, 20%, 25%, 30%, 40%, 50%, 55%, 60%, 65%, 70%, 75%, 80%, 90%, 95%, 97.5%, 99% or more reduction) or a complete elimination of the presence, or alternatively the accumulation, of one or more pathological, clinical, or biological markers that are associated with ALS. For example, in some embodiments, upon administration to a subject, a pharmaceutical composition described herein demonstrates or achieves a reduction in muscle loss, muscle twitching, muscle weakness, spasticity, abnormal tendon reflexes, Babinski sign, breathing problems, facial weakness, slurred speech, loss of perception, loss of reasoning, loss of judgment, and/or loss of imagination.

In some embodiments, treatment refers to increased survival (e.g., survival time). For example, treatment can result in an increased life expectancy of a patient. In some embodiments, treatment results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with ALS without treatment. In some embodiments, treatment results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with ALS without treatment. In some embodiments, treatment results in long term survival of a patient. As used herein, the term “long term survival” refers to a survival time or life expectancy longer than about 40 years, 45 years, 50 years, 55 years, 60 years, or longer.

The term “improve,” “increase” or “reduce,” as used herein, indicates values that are relative to a control. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with ALS, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having ALS or having the potential to develop ALS. In some instances, a subject to be treated is genetically predisposed to developing ALS. For example, a subject to be treated has a mutation in a SOD1 gene, ALS2 gene, VAPB gene, SETX gene, TDP-43 gene, FUS/TLS gene, and/or OPTN gene.

### Combination Therapy

In some embodiments, an NMD agent described herein is administered to a subject in combination with one or more additional therapies to treat ALS or one or more symptoms of ALS. For example, an NMD agent can be administered in combination with riluzole (Rilutek®, Sanofi-Aventis, Bridgewater, NJ), baclofen, diazepam, trihexyphenidyl or amitriptyline.

In some embodiments, combined administration of an NMD agent and a second agent results in an improvement in ALS or a symptom thereof to an extent that is greater than one produced by either the NMD agent or the second agent alone. The difference between the combined effect and the effect of each agent alone can be a statistically significant difference.

In some embodiments, combined administration of an NMD agent and a second agent allows administration of the second agent at a reduced dose, at a reduced number of doses, and/or at a reduced frequency of dosage compared to a standard dosing regimen approved for the second agent. For example, approved standard regimen for Rilutek® is 50 mg every 12 hours. Accordingly, for administration in combination with an NMD agent, a therapeutically effective amount of Rilutek® can be a dosage of less than about 50 mg and/or a frequency of greater than about every 12 hours.

In some embodiments, an immunosuppressant agent known to the skilled artisan can be administered to a subject in combination with an NMD polypeptide described herein. Exemplary immunosuppressant agents include, without limitation, cyclosporine, FK506, rapamycin, CTLA4-Ig, anti-TNF agents (such as etanercept), daclizumab (e.g., Zenapax™), anti-CD2 agents, anti-CD4 agents, and anti-CD40 agents.

### Methods of Identifying Modulators of NMD Polypeptide Expression or Activity

NMD polypeptides described herein (e.g., UPF1, UPF2, UPF3, SMG1, SMG5, SMG6, or SMG7 polypeptides) are useful for identifying agents that can be potentially used to treat ALS. For example, an agent that increases expression or activity of an NMD polypeptide can be identified as an agent that can be used to treat ALS. Numerous methods exist for evaluating whether an agent alters NMD polypeptide expression or NMD polypeptide activity or level. In one embodiment, the ability of a test agent to modulate (e.g., increase or decrease) (e.g., permanently or temporarily) expression from an NMD polynucleotide promoter is evaluated by e.g., routine reporter (e.g., LacZ, luciferase, or GFP) transcription assay. For example, a cell or transgenic animal whose genome comprises a reporter gene operably linked to an NMD polynucleotide promoter, can be contacted with a test agent, and the ability of the test agent to increase or decrease reporter activity is indicative of the ability of the agent to modulate an NMD polypeptide.

In some embodiments, effects of a test agent on NMD polypeptide expression or NMD polypeptide activity or level can be evaluated in a cell, cell lysate, or subject, preferably a non-human experimental mammal, and more preferably a rodent (e.g., a rat, mouse, rabbit), or explant thereof. Methods of assessing NMD polypeptide expression are well know in the art, e.g., Northern analysis, ribonuclease protection assay, reverse transcription-polymerase chain reaction (RT-PCR) or RNA in situ hybridization (see, e.g., Sambrook et al. Molecular Cloning: A Laboratory Manual (3^{rd} ed. 2001)). The level of NMD polypeptide can be monitored by, e.g., Western analysis, immunoassay, or in situ hybridization. In some embodiments, a DNA construct encoding an NMD polypeptide/GFP fusion protein is transfected into cells, and level of GFP fluorescence in the presence or absence of a test agent is determined. An increase in fluorescence in the presence of the test agent is indicative of the ability of the test agent to increase NMD polypeptide level.

In some embodiments, the effect of a test agent on NMD polypeptide expression or NMD polypeptide activity or level is confirmed in a second assay, e.g., is observed as a change, in the presence of the test agent, in the ability of the NMD polypeptide to reduce toxicity of a cell, e.g., a neuronal cell, expressing TDP-43 and/or FUS.

Agents and test agents to be used in the methods described herein include crude or partially or substantially purified extracts of organic sources, e.g., botanical (e.g., herbal) and algal extracts, inorganic elements or compounds, as well as partially or substantially purified or synthetic agents, e.g., small molecules, polypeptides, antibodies, and polynucleotides, and libraries of these.

In one example, combinatorial chemical libraries can be produced or obtained that sample chemical compounds that are structurally or chemically related or unrelated. Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493 (1991); and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries, peptide nucleic acid libraries (see, e.g., U.S. Pat. No. 5,539,083), antibody libraries (see, e.g., Vaughn et al., Nature Biotechnology, 14 (3): 309-314 (1996) and PCT/US96/10287), carbohydrate libraries (see, e.g., Liang et al., Science, 274:1520-1522 (1996) and U.S. Pat. No. 5,593,853), and small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337;benzodiazepines, U.S. Pat. No. 5,288,514, and the like).

The invention is further illustrated by the following examples. The examples are provided for illustrative purposes only. They are not to be construed as limiting the scope or content of the invention in any way.

### EXAMPLES

### Example 1

### Expression of UPF1 in Neurons Eliminates Toxicity of FUS or TDP-43

The present Example describes reduction of TDP-43 or FUS-mediated neuronal toxicity by UPF1.

A yeast model of ALS was used to identify a human gene, UPF1, which suppressed toxicity of FUS/TLS in yeast (Ju et al., PLoS Biol. 9:e1001052 (2011)). Further, UPF1 was able to suppress the cytotoxicity of ALS-associated TDP-43 mutations in yeast as well.

To test the efficacy of UPF1 in reducing TDP-43 or FUS-mediated cytotoxicity in neurons, UPF1 was expressed in motor neurons expressing disease-associated FUS or TDP-43. Motor neurons were either isolated from mice or created from fibroblasts taken from human ALS patients using iPS cell techniques (described in Yamanaka et al., Cell 126:663-676 (2006)). FUS or TDP-43 were tagged with EGFP (Enhanced Green Fluorescent Protein) and expressed in motor neurons, which were visualized by fluorescent microscopy using mApple.

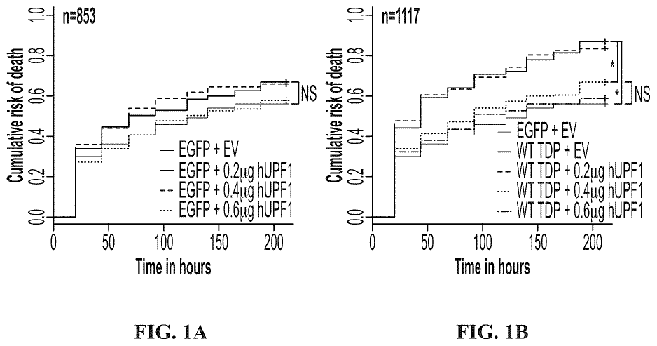

The motor neurons died within a few days of FUS or TDP-43 expression due to toxicity of these ALS-related proteins. UPF1 was expressed in the motor neurons and Kaplan-Meyer survival curves were determined. As shown in FIG. 1A, UPF1 expression had no effect on survival of wild type neurons, indicating that UPF1 was not a generic survival factor. However, as shown in FIG. 1B, UPF1 was able to completely eliminate the toxicity of TDP-43 in a dose-dependent manner. UPF1 had a similar effect on cells expressing FUS (data not shown). Moreover, UPF1 expression was unable to rescue the toxicity of ALS-associated mutants of SOD1, demonstrating for the first time that SOD1-dependent fALS is a distinct disease mechanistically.

### Example 2

### Yeast Screening Assay for Compounds That Rescue FUS Toxicity

A drug screen based on the yeast model described in Example 1 was developed to identify compounds that rescue toxicity that resulted from FUS expression. Because the phenotype was rescue from cell death, the screen demonstrated exceptionally good signal-to noise, with a Z′ score of around 0.8.

Briefly, two yeast strains were engineered: “1XFUS”, in which a FUS gene was stably integrated at the HIS locus; and “1XVec”, in which an empty vector was integrated at the same locus. The media used were YPRaffinose and 2XYPGalactose (2× concentrated). Yeast cells were grown by inoculating a single colony of 1XFUS strain or 1XVec strain into 2 ml YPRaffinose medium and were grown overnight at 30° C. The overnight cultures were then used to inoculate 50 ml YPRaffinose medium at OD600=0.2 and were grown for 24 hrs at 30° C.

The cultures were then diluted in 500 ml 2× YPGalactose medium at OD600=0.2. 384 well plates were pre-filled with 25 μl of each test compound at a concentration of 30 μM. A Multidrop was used to add 25 μl of the suspension of 1XFUS to each well on columns 1-23 of the plate; 1XVec was added to each well on column 24 as control. The yeast and compounds were mixed thoroughly. The plates were kept in a humidified incubator at 30° C. The OD600 of each plate was monitored at 24 hr and 48 hrs.

The compound(s) that rescued the growth of 1XFUS were selected and retested. The compounds that passed the retest were further checked in a 10-dose response experiment. The compounds that demonstrated good dose responses were re-ordered, and retested.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Sequences

```
(SEQ ID NO: 1)
UPF1 nuclcotide sequence (GenBank Accession No. U59323.1, nt 176-3532)
    176 atgag
    181 cgtggaggcg tacgggccca gctogcagac tctcactttc ctggacacgg aggaggccga
    241 gctgcttggc gccgacacac agggctccga gttcgagttc accgacttta ctcttcctag
    301 ccagacgcag acgccccccg gcggccccgg cggcccgggc ggtggoggcg cgggaagccc
    361 gggcggcgcg ggcgccggcg ctgcggcggg acagctcgac gcgcaggttg ggcccgaagg
    421 catcctgcag aacggggctg tggacgacag tgtagccaag accagccagt tgttggctga
    481 gttgaacttc gaggaagatg aagaagacac ctattacacg aaggacatcc ccatacacgc
    541 ctgcagttac tgtggaatac acgatcctgc ctgcgtggtt tactgtaata ccagcaagaa
    601 gtggttctgc aacggacgtg gaaatacttc tggcagccac attgtaaatc accttgtgag
    661 ggcaaaatgc aaagaggtga ccctgcacaa ggacgggccc ctgggggaga cagtcctgga
    721 gtgctacaac tgcggctgtc gcaacgtctt cctcctcggc ttcatcccgg ccaaagctga
    781 ctcagtggtg gtgctgctgt gcaggcagcc ctgtgccagc cagagcagcc tcaaggacat
    841 caactgggac agctcgcagt ggcagccgct gatccaggac cgctgcttcc tgtcctggct
    901 ggtcaagatc ccctccgagc aggagcagct gcgggcacgc cagatcacgg cacagcagat
    961 caacaagctg gaggagctgt ggaaggaaaa cccttatgcc acgctggagg acctggagaa
  1021 gccgggggtg gacgaggagc cgcagcatgt cctcctgcgg tacgaggacg cctaccagta
  1081 ccagaacata ttcgggcccc tggtcaagct ggaggccgac tacgacaaga agctgaagga
  1141 gtcccagact caagataaca tcactgtcag gtgggacctg ggccttaaca agaagagaat
  1201 cgcctacttc actttgccca agactgactc tgacatgcgg ctcatgcagg gggatgagat
  1261 atgcctgcgg tacaaagggg accttgcgcc cctgtggaaa gggatcggcc acgtcatcaa
  1321 ggtccctgat aattatggcg atgagatcgc cattgagctg cggagcagcg tgggtgcacc
  1381 tgtggaggtg actcacaact tccaggtgga ttttgtgtgg aagtcgacct cctttgacag
  1441 gatgcagagc gcattgaaaa cgtttgccgt ggatgagacc tcggtgtctg gctacatcta
  1501 ccacaagctg ttgggccacg aggtggagga cgtaatcacc aagtgccagc tgcccaagcg
  1561 cttcacggcg cagggcctcc ccgacctcaa ccactcccag gtttatgccg tgaagactgt
  1621 gctgcaaaga ccactgagcc tgatccaggg cccgccaggc acggggaaga cggtgacgtc
  1681 ggccaccatc gtctaccacc tggcccggca aggcaacggg ccggtgctgg tgtgtgctcc
  1741 gagcaacatc gccgtggacc agctaacgga gaagatccac cagacggggc taaaggtcgt
  1801 gcgcctctgc gccaagagcc gtgaggccat cgactccccg gtgtcttttc tggccctgca
  1861 caaccagatc aggaacatgg acagcatgcc tgagctgcag aagctgcagc agctgaaaga
  1921 cgagactggg gagctgtcgt ctgccgacga gaagcggtac cgggccttga agcgcaccgc
  1981 agagagagag ctgctgatga acgcagatgt catctgctgc acatgtgtgg gcgccggtga
  2041 cccgaggctg gccaagatgc agttccgctc cattttaatc gacgaaagca cccaggccac
  2101 cgagccggag tgcatggttc ccgtggtcct cggggccaag cagctgatcc ttgtaggcga
  2161 ccactgccag ctgggcccag tggtgatgtg caagaaggcg gccaaggccg ggctgtcaca
  2221 gtcgctcttc gagcgcctgg tggtgctggg catccggccc atccgcctgc aggtccagta
  2281 ccggatgcac cctgcactca gcgccttccc atccaacatc ttctacgagg gctccctcca
  2341 gaatggtgtc actgcagcgg atcgtgtgaa gaagggattt gacttccagt ggccccaacc
  2401 cgataaaccg atgttcttct acgtgaccca gggccaagag gagattgcca gctcgggcac
  2461 ctcctacctg aacaggaccg aggctgcgaa cgtggagaag atcaccacga agttgctgaa
  2523 ggcaggcgcc aagccggacc agattggcat catcacgccc tacgagggcc agcgctccta
  2581 cctggtgcag tacatgcagt tcagcggctc cctgcacacc aagctctacc aggaagtgga
  2641 gatcgccagt gtggacgcct ttcagggacg cgagaaggac ttcatcatcc tgtcctgtgt
  2701 gcgggccaac gagcaccacg gcattggctt tttaaatgac cccaggcgtc tgaacgtggc
  2761 cctgaccaga gcaaggtatg gcgtcatcat tgtgggcaac ccgaaggcac tatcaaagca
  2821 gccgctctgg aaccacctgc tgaactacta taaggagcag aaggtgctgg tggaggggcc
  2881 gctcaacaac ctgcgtgaga gcctcatgca gttcagcaag ccacggaagc tggtcaacac
  2941 tatcaacccg ggagcccgct tcatgaccac agccatgtat gatgcccggg aggccatcat
  3001 cccaggctcc gtctatgatc ggagcagcca gggccggcct tccagcatgt acttccagac
  3061 ccatgaccag attggcatga tcagtgccgg ccctagccac gtggctgcca tgaacattcc
  3121 catccccttc aacctggtca tgccacccat gccaccgcct ggctattttg gacaagccaa
  3181 cgggcctgct gcagggcgag gcaccccgaa aggcaagact ggtcgtgggg gacgccagaa
  3241 gaaccgcttt gggcttcctg gacccagcca gactaacctc cccaacagcc aagccagcca
  3301 ggatgtggcg ccacagccct tctctcaggg cgccctgacg cagggctaca tctccatgag
  3361 ccagccttcc cagatgagcc agcccggcct ctcccagccg gagctgtccc aggacagtta
  3421 ccttggtgac gagtttaaat cacaaatcga cgtggcgctc tcacaggact ccacgtacca
  3481 gggagagcgg gcttaccagc atggcggggt gacggggctg tcccagtatt aa
(SEQ ID NO: 2)
UPF1 amino acid sequence (GenBank Accession No. AAC51140.1)
        1 msveaygpss qtltfldtee aellgadtqg sefeftaftl psqtqtppgg pggpggggag
      61 spggagagaa agqldaqvgp cgilqngavd dsvaktsqll aelnfeedee dtyytkdlpi
    121 hacsycgihd pacvvycnts kkwfcngrgn tsgshivnhl vrakckcvtl hkdgplgetv
    181 lecyncgcrn vfllgfipak adsvvvllcr qpcasqsslk dinwdssqwq pliqdrcfls
    241 wlvkipseqe qlrarqitaq qinkleelwk enpsatledl ekpgvdeepg hvllryeday
    301 qyqnifgplv kleadydkkl kesqtqdnit vrwdlglnkk riayftlpkt dsdmrlmqgd
    361 eiclrykgdl aplwkgighv ikvpdnygde iaielrssvg apvevthnfg vdfvwkstsf
    421 drmqsalktf avdetsvsgy iyhkllghcv edvitkcqlp krftaqglpd lnhsqvyavk
    481 tvlqrplsli qgppgtgktv tsativyhla rqgngpvlvc apsniavdql tekihqtglk
    541 vvrlcaksre aidspvsfla lhnqirnmds mpelqklqql kdetgelssa dekryralkr
    601 taerellmna dvicctcvga gdprlakmgf rsilidestq atepecmvpv vlgakqlilv
    661 gdhcqlgpvv mckkaakagl sqslferlvv lgirpirlqv qyrmhpalsa fpsnifyegs
    721 lqngvtaadr vkkgfdfqwp qpdkpmffyv tqgqeeiass gtsylnrtca anvckittkl
    781 lkagakpdgi giitpyeqgr sylvgymgfs gslhtklyge veiasvdafg grekdfiils
    841 cvranehqgi gflndprrln valtrarygv iivgnpkals kqplwnhlln yykeqkvlvc
    901 gplnnlrcsl mgfskprklv ntinpgarfm ttamydarea iipgsvydrs sqgrpssmyf
    961 qthdqigmis agpshvaamn ipipfnlvmp pmpppgyfgq angpaagrgt pkgktgrggr
  1021 qknrfglpgp sqtnlpnsqa sqdvasqpfs qgaltqgyis msqpsqmsqp glsqpelsqd
  1081 sylgdefksq idvalsqdst yqgerayqhg gvtglsqy
(SEQ ID NO: 3)
UPF2 nucleotide sequence (GenBank Accession No. AF318574.1) (nt 76-3894)
      76 atgcc agctgagcgt aaaaagccag caagtatgga agaaaaagac
    121 tctttaccaa acaacaagga aaaagactgc agtgaaaggc ggacagtgag cagcaaggag
    181 aggccaaaag acgatatcaa gctcactgcc aagaaggagg tcagcaaggc ccctgaagac
    241 aagaagaaga gactggaaga tgataagaga aaaaaggaag acaaggaacg caagaaaaaa
    301 gacgaagaaa aggtgaaggc acaggaagaa tcaaagaaaa aagaagagga agaaaaaaag
    361 aaacatcaag aggaagagag aaagaagcaa gaagagcagg ccaaacgtca gcaagaagaa
    421 gaagcagctg ctcagatgaa agaaaaagaa gaatccattc agcttcatca ggaagcttgg
    481 gaacgacatc atttaagaaa ggaacttcgt agcaaaaacc aaaatgctcc ggacagccga
    541 ccagaggaaa acttcttcag ccgcctcgac tcaagtctga agaaaaatac tgcttttgtc
    601 aagaaactaa aaactattac agaacaacag agagactoct tgtcccatga ttttaatggc
    661 ctaaatttaa gcaaatacat tgcagaagct gtagcttcca tcgtggaagc aaaactaaaa
    721 atctctgatg tgaactgtgc tgtgcacctc tgctctctct ttcaccagcg ttatgctgac
    781 tttgccccat cacttottca ggtctggaaa aaacattttg aagcaaggaa agaggagaaa
    841 acacctaaca tcaccaagtt aagaactgat ttgcgtttta ttgcagaatt gacaatagtt
    901 gggattttca ctgacaagga aggtctttcc ttaatctatg aacagctaaa aaatattatt
    961 aatgctgatc gggagtccca cactcatgtc tctgtagtga ttagtttctg tcgacattgt
  1021 ggagatgata ttgctggact tgtaccaagg aaagtaaaga gtgctgcaga gaagtttaat
  1081 ttgagttttc ctcctagtga gataattagt ccagagaaac aacagccctt ccagaatctt
  1141 ttaaaagagt actttacgtc tttgaccaaa cacctgaaaa gggaccacag ggagctccag
  1201 aatactgaga gacaaaacag gcgcattcta cattctaaag gggagctcag tgaagataga
  1261 cataaacagt atgaggaatt tgctatgtct taccagaagc tgctggcaaa ttctcaatcc
  1321 ttagcagacc ctttggatga aaatatgcca gatcttcctc aagacaaacc cacaccagaa
  1381 gaacatgggc ctggaattga tatattcaca cctggtaaac ctggagaata tgacttggaa
  1441 ggtggtatat gggaagatga agatgctcgg aatttttatg agaacctcat tgatttgaag
  1501 gcttttgtcc cagccatctt gtttaaagac aatgaaaaaa gttgtcagaa taaagagtcc
  1561 aacaaagatg ataccaaaga ggcaaaagaa tctaaggaga ataaggaggt atcaagtccc
  1621 gatgatttgg aacttgagtt ggagaatcta gaaattaatg atgacacctt agaattagag
  1681 ggtggagatg aagctgaaga tottacaaag aaacttcttg atgaacaaga acaagaagat
  1741 gaggaagcca gcactggatc tcatctcaag ctcatagtag atgctttcct acagcagtta
  1801 cccaactgtg tcaaccgaga tctgatagsc aaggcagcaa tggatttttg catgaacatg
  1861 aacacaaaag caaacaggaa gaagttggta cgggcactct tcatagttcc tagacaaagg
  1921 ttggatttgc taccatttta tgcaagattg gttgctacat tgcatccctg catgtctgat
  1981 gtagcagagg atctttgttc catgctgagg ggggatttca gatttcatgt acggaaaaag
  2041 gaccagatca acattgaaac aaagaataaa actgttcgtt ttataggaga actaactaag
  2101 tttaagatgt tcaccaaaaa tgacacactg cattgtttaa agatgcttct gtcagacttc
  2161 tctcatcacc atattgaaat ggcatgcacc ctgctggaga catgtggacg gtttcttttc
  2221 agatctccag aatctcacct gaggaccagt gtacttttgg agcaaatgat gagaaagaag
  2281 caagcaatgc atcttgatgc gagatacgtc acaatggtag agaatgcata ttactactgc
  2341 aacccacctc cagctgaaaa aaccgtgaaa aagaaacgtc ctcctctcca ggaatatgtc
  2401 cggaaacttt cgcacaaaca tctctctaag gttaccaccg agaaggtttt gagacagatg
  2461 cgaaagctgc cctggcagga ccaagaagtg aaagactatg ttatttgttg tatgataaac
  2521 atctggaatg tgaaatataa tagtattcat tgtgtagcca acctcttagc aggactagtg
  2581 ctctaccaag aggatgttgg gatccacgtt gtggatggag tgttagaaga tattcgatta
  2641 ggaatggagg ttaatcaacc taaatttaat cagaggcgca tcagcagtgc caagttctta
  2701 ggagaacttt acaattaccg aatggtggaa toagctgtta ttttcagaac tctgtattct
  2761 tttacctcat ttggtgttaa toctgatggc totccaagtt ccctggaccc acctgagcat
  2821 cttttcagaa ttagactogt atgcactatt ctggacacat gtggccagta ctttgacaga
  2881 ggttccagta aacgaaaact tgattgtttc cttgtatatt ttcagogtta tgtttggtgg
  2941 aagaaaagtt tggaggtttg gacaaaagac catccatttc ctattgatat agattacatg
  3001 atcagtgata cactagaact gctaagacca aagatcaaac tctgtaattc tctggaagaa
  3061 tccatcaggc aggtacaaga cttggaacga gaattcttaa taaaactagg cctagtaaat
  3121 gacaaagact caaaagattc tatgacagaa ggagaaaatc ttgaagagga tgaagaagaa
  3181 gaagaaggtg gggctgaaac agaagaacaa tctggaaatg aaagtgaagt aaatgagcca
  3241 gaagaagagg agggttctga taatgatgat gatgagggag aagaagagga ggaagagaat
  3301 acagattacc ttacagattc caataaggaa aatgaaacog atgaagagaa tactgaggta
  3361 atgattaaag gcggtggact taagcatgta ccttgtgtag aagatgagga cttcattcaa
  3421 gctctggata aaatgatgct agaaaatcta cagcaacgaa gtggtgaatc tgttaaagtg
  3481 caccaactag atgtggccat tcctttgcat ctcaaaagcc agctgaggaa agggccccca
  3541 ctgggaggtg gggaaggaga ggctgagtct gcagacacaa tgccgtttgt catgttaaca
  3601 agaaaaggca ataaacagca gtttaagatc cttaatgtac ccatgtcctc tcaacttgct
  3601 gcaaatcact ggaaccagca acaggcagaa caagaagaga ggatgagaat gaagaagctc
  3721 acactagata tcaatgaacg gcaagaacaa gaagattatc aagaaatgtt gcagtctctt
  3781 gcacagcgcc cagctccagc aaacaccaat cgtgagaggc ggcctcgcta ccaacatccg
  3841 aagggagcac ctaatgcaga tctaatcttt aagactggtg ggaggagacg ttga
(SEQ ID NO: 4)
UPF2 amino acid sequence (GenBank Accession No. AAG60689.1)
        1 mpaerkkpas meekdslpnn kekdcserrt vsskerpkdd ikltakkevs kapedkkkrl
      61 eddkrkkedk erkkkdeekv kaeeeskkke eeekkkhqee erkkqeeqak rqqeeeaaaq
    121 mkekeesiql hgeawerhhl rkelrsknqn apdsrpeenf fsrldsslkk ntafvkklkt
    181 iteqqrdsls hdfnglnlsk yiaeavasiv eaklkisdvn cavhlcslfh qryadtapsl
    24l lqvwkkhfea rkeektpnit klrtdlrfia eltivgiftd keglsliyeq lkniinadre
    301 shthvsvvis fcrhcgddia glvprkvksa aekfnlsfpp seiispekgg pfqnllkcyf
    361 tsltkhlkrd hrelqnterq nrrilhskge lsedrhkgye efamsyqkll ansqsladll
    421 denmpalpgd kptpeehgpg idiftpgkpg eydleggiwe dedarnfyen lidlkafvpa
    481 ilfkdneksc qnkesnkddt keakeskenk evsspddlel elenleindd tleleggdea
    541 edltkkllde qeqedeeast gshlklivda flqqlpncvn rdlidkaamd fcmnmntkan
    610 rkklvralfi vprqrldllp fyarlvatlh pcmsdvacdl csmlrgdfrf hvrkkdqini
    661 etknktvrfi geltkfkmft kndtlhclkm llsdfshhhi emactlletc grflfrspes
    721 hlrtsvlleg mmrkkqamhl daryvtmvcn ayyycnpppa ektvkkkrpp lqeyvrklly
    781 kdlskvttek vlrgmrklpw qdqevkdyvi ccminiwnvk ynsihcvanl laglvlyqed
    841 vgihvvdgvl edirlgmevn qpkfnqrris sakflgelyn yrmvesavif rtlysftsfg
    901 vnpdgspssl dppchlfrir lvctildtcg qyfdrgsskr kldcflvyfg ryvwwkksle
    961 vwtkdhpfpi didymisdtl ellrpkiklc nsleesirqv qdlereflik lglvndkask
  1021 dsmtegenle edeeeeegga eteeqsgnes evnepeeeeg sdndddegee eeeentdylt
  1081 dsnkenedce entevmikgg glkhvpcved edfiqaldkm mlenlqqrsg esvkvhqldv
  1141 aiplhlksql rkgpplggge geaesadtmp fvmltrkgnk qqfkilnvpm ssqlaanhwn
  1201 qqqaeqeerm rmkkltldin erqeqedyqe mlqslaqrpa pantnrerrp ryqhpkgapn
  1261 adlifktggr rr
(SEq ID NO: 5)
UPF3 nucleotide sequence (GenBank Accession No. AF318575_1 )(nt 22-1380)
      22 atgctgtcg gccctagaag tgcagttcca ccgcgactcg
      61 cagcagcagg aggctgagac gccgccaact tcgtcctccg gttgcggggg cggtgcgggc
    121 aaacctcgcg aggagaagag gacggccctg agcaaggtgg tcatccgccg cctgcctcog
    181 ggcctcacca aggagcagct ggaggagcag ctgcgcccgc tgccagcaca cgactacttc
    241 gagttcttcg cagccgacct gagtctttat cctcatctct actcaagagc atacattaat
    301 tttaggaatc ctgatgacat ccttcttttt agagatcgtt ttgatggata tatcttcctt
    361 gacagcaaag gcctacaata tcctgcagtg gtagagtttg ctccattcca gaagatagcc
    421 aaaaagaagc tgagaaaaaa agatgccaag actggaagca tcgaagatga tccagaatat
    481 aagaagtttt tagaaaccta ctgtgtggag gaagagaaga ccagtgccaa ccctgagact
    541 ctgctggggg agatggaggc gaagacaaga gagctcattg ctagaagaac cacacctctt
    601 ttggaatata ttaaaaatag aaaattagaa aagcagagaa ttcgagaaga gaagcgagaa
    661 gaacggagga ggagagagtt agaaaagaaa cgtttgcggg aagaggaaaa aagaagaaga
    721 agagaagaag aaagatgcaa aaaaaaagag acagataaac agaagaaaat tgcagagaaa
    781 gaagtaagga ttaagcttct taagaaacca gaaaagggag aggaaccaac cacagagaaa
    841 ccaaaagaaa gaggagagga gattgatact ggaggtggca agcaggaatc ctgtgccccc
    901 ggtgcagtcg taaaagccag gcccatggaa ggctcgctgg aggagcccca ggagacgtca
    961 cacagcggca gtgataaaga gcacagggat gtggagagat ctcaagaaca agaatctgaa
  1021 gcacaaagat accatgtgga tgacggcagg aggcacagag ctcaccacga gcctgaacgg
  1081 ctttccagaa ggagtgagga tgagcagaga tgggggaaag gacctggcca agacagaggg
  1141 aagaagggga gocaggacag cggggctccg ggggaggcca tggagagact gggaagagcg
  1201 caaaggtgtg acgacagtcc agcacccaga aaagagcgac tggcaaacaa ggaccggcca
  1261 gccttgcagc tgtatgatcc aggagctcgc ttccgagcgc gagagtgtgg cggaaacagg
  1321 aggatctgca aggcagaagg ttcggggact ggtcctgaga agagggaaga ggcagagtga
(SEQ ID NO: 6)
UPF3 amino acid sequence (GenBank Accession No. AAG60690.1)
        1 mlsalevgfh rdsqqqeaet pptsssgcgg gagkpreekr talskvvirr lppgltkeql
      61 eeqlrplpah dyfeffaadl slyphlysra yinfrnpddi llfrdrfdgy ifldskgley
    l21 pavvefapfq kiakkklrkk daktgsiedd peykkflety cveeektsan pctllgemea
    181 ktreliarrt tplleyiknr klekqriree kreerrrrel ekkrlreeek rrrreeerck
    241 kketdkgkki aekevrikll kkpekgeept tekpkergee idtgggkges capgavvkar
    301 pmegsleepg etshsgsdke hrdversqeq eseaqryhvd dgrrhrahhr perlsrrsed
    361 eqrwgkgpgq drgkkgsqds gapgeamerl graqrcddsp aprkerlank drpalqlydp
    421 garfrarecg gnrrickaeg sgtgpekree ae
(SEQ ID NO: 7)
SMG1 nucleotide sequence (GenBank Accession No. NM_015092.4. nt 364-11349)
    364 atgagcc gcagagcccc ggggtctcgg ctgagcagcg gcggcggcgg cggoggcacc
    421 aagtatccgc ggagctggaa tgactggcaa cccagaactg atagtgcatc agccgaccca
    481 gataatttaa aatattcttc atccagagat agaggtggtt cttcctctta tggactgcaa
    541 ccttcaaatt cagctgtggt gtctcggcaa aggcacgatg ataccagagt ccacgctgac
    601 atacagaatg acgaaaaggg tggctacagt gtcaatggag gatctgggga aaatacttat
    661 ggtcggaagt cgttggggca agagctgagg gttaacaatg tgaccagccc tgagttcacc
    721 agtgttcagc atggcagtcg tgctttagcc accaaagaca tgaggaaatc acaggagaga
    781 tcgatgtctt attctgatga gtctcgactg tcgaatcttc ttcggaggat cacccgggaa
    841 gacgacagag accgaagatt ggctactgta aagcagttga aagaatttat tcagcaacca
    901 gaaaataagc tggtactagt taaacaattg gataatatct tggctgctgt acatgacgtg
    961 cttaatgaaa gtagcaaatt gcttcaggag ttgagacagg agggagcttg ctgtcttggc
  1021 cttctttgtg cttctctgag ctatgaggct gagaagatct tcaagtggat ttttagcaaa
  1081 tttagctcat ctgcaaaaga tgaagttaaa ctcctctact tatgtgccac ctacaaagca
  1141 ctagagactg taggagaaaa gaaagccttt tcatctgtaa tgcagattgt aatgaccagc
  1201 ctgcagtcta ttcttgaaaa tgtggataca ccagaattgc tttgtaaatg tgttaagtgc
  1261 attcttttgg tggctcgatg ttaccctcat attttcagca ctaattttag ggatacagtt
  1321 gatatattag ttggatggca tatagatcat actcagaaac cttcgctcac gcagcaggta
  1381 tctgggtggt tgcagagttt ggagccattt tgggtagctg atcttgcatt ttctactact
  1441 cttcttggtc agtttctgga agacatggaa gcatatgctg aggacctcag ccatgtggcc
  1501 tctggggaat cagtggatga agatgtccct cctccatcag tgtcattacc aaagctggct
  1561 gcacttctcc gggtatttag tactgtggtg aggagcattg gggaacgctt cagcccaatt
  1621 aggggtcctc caattactga ggcatatgta acagatgttc tgtacagagt aatgagatgt
  1681 gtgacggctg caaaccaggt gtttttttct gaggctgtgt tgacagctgc taatgagtgt
  1741 gttggtgttt tgctaggcag cttggatcct agcatgacta tacattgtga catggtcatt
  1801 acatatggat tagaccaact ggagaattgc cagacttgtg gtaccgatta tatcatctca
  1861 gtcttgaatt tactcacgct gattgttgaa cagataaata cgaaactgcc atcatcattt
  1921 gtagaaaaac tgtttatacc atcatctaaa ctactattct tgcgttatca caaagaaaaa
  1981 gaggttgttg ctgtagccca tgctgtttat caagcagtgc tcagcttgaa gaatattcct
  2041 gttttggaga ctgcctataa gttaatattg ggagaaatga cttgtgccct aaacaacctc
  2101 ctacacagtc tacaacttcc tgaggcctgt tctgaaataa aacatgaggc ttttaagaat
  2161 catgtgttca atgtagacaa tgcaaaattt gtagttatat ttgacctcag tgccctgact
  2221 acaattggaa atgccaaaaa ctcactaata gggatgtggg cgctatctcc aactgtcttt
  2281 gcacttctga gtaagaatct gatgattgtg cacagtgacc tggctgttca cttccctgcc
  2341 attcagtatg ctgtgctcta cacattgtat tctcattgta ccaggcatga tcactttatc
  2401 tctagtagcc tcagttcttc ctctccttct ttgtttgatg gagctgtgat tagcactgta
  2461 actacggcta caaagaaaca tttctcaatt atattaaatc ttctgggaat attacttaag
  2521 aaagataacc ttaaccagga cacgaggaaa ctgttaatga cttgggcttt ggaagcagct
  2581 gttttaatga agaagtctga aacatacgca cctttattct ctcttccgtc tttccataaa
  2641 ttttgcaaag gccttttagc caacactctc gttgaagatg tgaatatctg tctgcaggca
  2701 tgcagcagtc tacatgctct gtcctcttcc ttgccagatg atcttttaca gagatgtgtc
  2761 gatgtttgcc gtgttcaact agtgcacagt ggaactcgta ttcgacaagc atttggaaaa
  2821 ctgttgaaat caattccttt agatgttgtc ctaagcaata acaatcacac agaaattcaa
  2881 gaaatttctt tagcattaag aagtcacatg agtaaagcac caagtaatac attccacccc
  2941 caagatttct ctgatgttat tagttttatt ttgtatggga actctcatag aacagggaag
  3001 gacaattggt tggaaagact gttctatagc tgccagagac tggataagcg tgaccagtca
  3061 acaattccac gcaatctcct gaagacagat gctgtccttt ggcagtgggc catatgggaa
  3121 gctgcacaat tcactgttct ttctaagctg agaaccccac tgggcagagc tcaagacacc
  3181 ttccagacaa ttgaaggtat cattcgaagt ctcgcagctc acacattaaa ccctgatcag
  3241 gatgttagtc agtggacaac tgcagacaat gatgaaggcc atggtaacaa ccaacttaga
  3301 cttgttcttc ttctgcagta tctggaaaat ctggagaaat taatgtataa tgcatacgag
  3361 ggatgtgcta atgcattaac ttcacctccc aaggtcatta gaactttttt ctataccaat
  3421 cgccaaactt gtcaggactg gctaacgcgg attcgactct ccatcatgag ggtaggattg
  3481 ttggcaggcc agcctgcagt gacagtgaga catggctttg acttgcttac agagatgaaa
  3541 acaaccagcc tatctcaggg gaatgaattg gaagtaacca ttatgatggt ggtagaagca
  3601 ttatgtgaac ttcattgtcc tgaagctata cagggaattg ctgtctggtc atcatctatt
  3661 gttggaaaaa atcttctgtg gattaactca gtggctcaac aggctgaagg gaggtttgaa
  3721 aaggcctctg tggagtacca ggaacacctg tgtgccatga caggcgttga ttgctgcatc
  3781 tccagctttg acaaatcggt gctcacctta gccaatgctg ggcgtaacag tgccagcccg
  3841 aaacattctc gtaatggtga atccagaaaa actgtgctgt ccaaaccgac tgactcttcc
  3901 cctgaggtta taaattattt aggaaataaa gcatgtgagt gctacatctc aattgccgat
  3961 tgggctgctg tgcaggaatg gcagaacgct atccatgact tgaaaaagag taccagtagc
  4021 acttccctca acctgaaagc tgacttcaac tatataaaat cattaagcag ctttgagtct
  4081 ggaaaatttg ttgaatgtac cgagcagtta gaattgttac caggagaaaa tatcaatcta
  4141 cttgctggag gatcaaaaga aaaaatagac atgaaaaaac tgcttcctaa catgttaagt
  4201 ccggatcoga gggaacttca gaaatccatt gaagttcaat tgttaagaag ttctgtttgt
  4261 ttggcaactg ctttaaaccc gatagaacaa gatcagaagt ggcagtctat aactgaaaat
  4321 gtggtaaagt acttgaagca aacatcccgc atogctattg gacctctgag actttctact
  4381 ttaacagttt cacagtcttt gccagttcta agtaccttgc agctgtattg ctcatctgct
  4441 ttggagaaca cagtttctaa cagactttca acagaggact gtottattco actcttcagt
  4501 gaagctttac gttcatgtaa acagcatgac gtgaggccat ggatgcaggc attaaggtat
  4561 actatgtacc agaatcagtt gttggagaaa attaaagaac aaacagtccc aattagaagc
  4621 catctcatgg aattaggtct aacagcagca aaatttgcta gaaaacgagg gaatgtgtcc
  4681 cttgcaacaa gactgctggc acagtgcagt gaagttcagc tgggaaagac caccactgca
  4741 caggatttag tccaacattt taaaaaacta tcaacccaag gtcaagtgga tgaaaaatgg
  4801 gggcccgaac ttgatattga adaaaccaaa ttgctttata cagcaggcca gtcaacacat
  4861 gcaatggaaa tgttgagttc ttgtgccata tctttctgca agtctgtgaa agctgaatat
  4921 gcagttgcta aatcaattct gacactggct aaatggatcc aggcagaatg gaaagagatt
  4981 tcaggacagc tgaaacaggt ttacagagct cagcaccaac agaacttcac aggtctttct
  5041 actttgtcta aaaacatact cactctaata gaactgccat ctgttaatac gatggaagaa
  5101 gagtatcctc ggatcgagag tgaatctaca gtgcatattg gagttggaga acctgacttc
  5161 attttgggac agttgtatca cctgtcttca gtacaggcac ctgaagtagc caaatcttgg
  5221 gcagcgttgg ccagctgggc ttataggtgg ggcagaaagg tggttgacaa tgccagtcag
  5281 ggagaaggtg ttcgtctgct gcctagagaa aaatctgaag ttcagaatct acttccagac
  5341 actataactg aggaagagaa agagagaata tatggtattc ttggacaggc tgtgtgtagg
  5401 ccggcgggga ttcaggatga agatataaca cttcagataa ctgagagtga agacaacgaa
  5461 gaagatgaca tggttgatgt tatctggcgt cagttgatat caagstgtcc atggctttca
  5521 gaacttgatg aaagtgcaac tgaaggagtt attaaagtgt ggaggaaagt tgtagataga
  5581 atattcagcc tgtacaaact ctcttgcagt gcatacttta ctttccttaa actcaacgct
  5641 ggtcaaattc ctttagatga ggatgaccct aggctgcatt taagtcacag agtggaacag
  5701 agcactgatg acatgattgt gatogccaca ttgcgcctgc tgcggttgct cgtgaagcat
  5761 gctggtgagc ttcggcagta tctggagcac ggcttggaga caacacccac tgcaccatgg
  5821 agaggaatta ttccgcaact tttctcacgc ttaaaccacc ctgaagtgta tgtgcgccaa
  5881 agtatttgta accttctctg ccgtgtggct caagattccc cacatctcat attgtatcct
  5941 gcaatagtgg gtaccatatc gcttagtagt gaatcccagg cttcaggaaa taaattttcc
  6001 actgcaattc caactttact tggcaatatt caaggagaag aattgctggt ttctgaatgt
  6061 gagggaggaa gtcctcctgc atctcaggat agcaataagg atgaacctaa aagtggatta
  6121 aatgaagacc aagccatgat gcaggattgt tacagcaaaa ttgtagataa gctgtcctct
  6181 gcaaacccca ccatggtatt acaggttcag atgctcgtgg ctgaactgcg cagggtcact
  6241 gtgctctggg atgagctctg gctgggagtt ttgctgcaac aacacatgta cgtcctgaga
  6301 cgaattcagc agcttgaaga tgaggtgaag agagtccaga acaacaacac cttacgcaaa
  6361 gaagagaaaa ttgcaatcat gagggagaag cacacagctt tgatgaagcc catcgtattt
  6421 gctttggagc atgtgaggag tatcacagcg gctcctgcag aaacacctca tgaaaaatgg
  6481 tttcaggata actatggtga tgccattgaa aatgccctag aaaaactgaa gactccattg
  6541 aaccctgcaa agoctgggag cagctggatt ccatttaaag agataatgct aagtttgcaa
  6601 cagagagcac agaaacgtgc aagttacatc ttgcgtcttg aagaaatcag tccatggttg
  6661 gctgccatga ctaacactga aattgctctt cctggggaag tctcagccag agacactgtc
  6721 acaatccata gtgtgggcgg aaccatcaca atcttaccga ctaaaaccaa gccaaagaaa
  6781 cttctctttc ttggatcaga tgggaagagc tatccttatc ttttcaaagg actggaggat
  6841 ttacatctgg atgagagaat aatgcagttc ctatctattg tgaataccat gtttgctaca
  6901 attaatcgcc aagaaacacc ccggttccat gctcgacact attctgtaac accactagga
  6961 acaagatcag gactaatcca gtgggtagat ggagccacac ccttatttgg tctttacaaa
  7021 cgatggcaac aacgggaagc tgccttacaa gcacaaaagg cccaagattc ctaccaaact
  7081 cctcagaatc ctggaattgt accccgtcct agtgaacttt attacagtaa aattggccct
  7141 gctttgaaaa cagttgggct tagcctggat gtgtcccgtc gggattggcc tcttcatgta
  7201 atgaaggcag tattggaaga gttaatggag gccacacccc cgaatctcct tgccaaagag
  7261 ctctggtcat cttgcacaac acctgatgaa tggtggagag ttacgcagtc ttatgcaaga
  7321 tctactgcag tcatgtctat ggttggatac ataattggcc ttggagacag acatctggat
  7381 aatgttctta tagatatgac gactggagaa gttgttcaca tagattacaa tgtttgcttt
  7441 gaaaaaggta aaagccttag agttcctgag aaagtacctt ttcgaatgac acaaaacatt
  7501 gaaacagcac tgggtgtaac tggagtagaa ggtgtattta ggctttcatg tgagcaggtt
  7561 ttacacatta tgcggcgtgg cagagagacc ctgctgacgc tgctggaggc ctttgtgtac
  7621 gaccctctgg tggactggac agcaggaggc gaggctgggt ttgctggtgc tgtctatggt
  7681 ggaggtggcc agcaggccga gagcaagcag agcaagagag agatggagcg agagatcacc
  7741 cgcagcctgt tttcttctag agtagctgag attaaggtga actggtttaa gaatagagat
  7801 gagatactgg ttgtgcttcc caagttggac ggtagcttag atgaatacct aagcttgcaa
  7861 gagcaactga cagatgtgga aaaactgcag ggcaaactac tggaggaaat agagtttcta
  7921 gaaggagctg aaggggtgga tcatccttct catactctgc aacacaggta ttctgagcac
  7981 acccaactac agaatcagca aagagctgtt caggaagcaa tccaggtgaa gctgaatgaa
  8041 tttgaacaat ggataacaca ttatcaggct gcattcaata atttagaagc aacacagctt
  8101 gcaagcttgc ctcaagagat aagcacacaa atggaoctto gtcctccaag ttacgtgcca
  8161 gcaacagcot ttctgcagaa tgctggtcag gcccacttga ttagccagtg cgagcagctg
  8221 gagggggagg ttggtgctct cctgcagcag aggcgotcog tgctccgtgg ctgtctggag
  8281 caactgcatc actatgcaac cgtggccctg cagtatccga aggccatatt tcagaaacat
  8341 cgaattgaac agtggaagac ctggatggaa gagctcatct gtaacaccac agtagagcgt
  8401 tgtcaagagc tctataggaa atatgaaatg caatatgctc cccagccacc cccaacagtg
  8461 tgtcagttca tcactgccac tgaaatgacc ctgcagcgat acgcagcaga catcaacagc
  8521 agacttatta gacaagtgga acgcttgaaa caggaagctg tcactgtgcc agtttgtgaa
  8581 gatcagttga aagaaattga acgttgcatt aaagttttcc ttcatgagaa tggagaagaa
  8641 ggatctttga gtctagcaag tgttattatt tctgcccttt gtacccttac aaggcgtaac
  8701 ctgatgatgg aaggtgcagc gtcaagtgct ggagaacagc tggttgatct gacttctcgg
  8761 gatggagcct ggttcttgga ggaactctgc agtatgagcg gaaacgtcac ctgcttggtt
  8821 cagttactga agcagcgcca cctggtgcca caggacttag atatcccgaa ccccatggaa
  8881 gcgtctgaga cagttcactt agccaatgga gtgtatacct cacttcagga attgaattcg
  8941 aatttccggc aaatcatatt tccagaagca cttcgatgtt taatgaaagg ggaatacacg
  9001 ttagaaagta tgctgcatga actggacggt cttattgagc agaccaccga tggcgttccc
  9061 ctgcagactc tagtggaatc tcttcaggcc tacttaagaa acgcagctat gggactggaa
  9121 gaagaaacac atgctcatta catcgatgtt gccagactac tacatgctca gtacggtgaa
  9181 ttaatccaac cgagaaatgg ttcagttgat gaaacaccca aaatgtcagc tggccagatg
  9241 cttttggtag cattcgatgg catgtttgct caagttgaaa ctgctttcag cttattagtt
  9301 gaaaagttga acaagatgga aattcccata gcttggcgaa agattgacat cataagggaa
  9361 gccaggagta ctcaagttaa tttttttgat gatgataatc accggcaggt gctagaagag
  9421 attttctttc taaaaagact acagactatt aaggagttct toaggctctg tggtaccttt
  9481 tctaaaacat tgtcaggatc aagttcactt gaagatcaga atactgtgaa tgggcctgta
  9541 cagattgtca atgtgaaaac cctttttaga aactottgtt tcagtgaaga ccaaatggcc
  9601 aaacctatca aggcattcac agctgacttt gtgaggcagc tattgatagg gctacccaac
  9661 caagccctcg gactcacacc gtgcagtttt accactactc tgggtgtaga catcattgct
  9721 caagtagagg caaaggactt tggtgccgaa agcaaagttt ctgttgatga tctctgtaag
  9781 aaagcggtgg aacataacat ccagataggg aagttctctc agctggttat gaacasgggc
  9841 actgtgttag caagttctta cgacactgcc tggaagaagc atgacttggt gcgaaggcta
  9901 gaaaccagta tttcttcttg taagacaagc ctgcagcggg ttcagctgca tattgccatg
  9961 tttcagtggc aacatgaaga tctacttatc aatagaccac aagccatgtc agtcacacct
10021 cccccacggt ctgctatoct aaccagcacg aaaaagaagc tgcataccct gagccagatt
10081 gaaacttcta ttgcaacagt tcaggagaag ctagctgcac ttgaatcaag tattgaacag
10141 cgactcaagt gggcaggtgg tgccaaccct gcattggccc ctgtactaca agattttgaa
10201 gcaacgatag ctgaaagaag aaatcttgtc cttaaagaga gccaaagagc aagtcaggtc
10261 acatttctct gcagcaatat cattcatttt gasagtttac gaacaagaac tgcagaagcc
10321 ttaaacctgg atgcggcgtt atttgaacta atcaagcgat gtcagcagat gtgttcgttt
10381 gcatcacagt ttaacagttc agtgtctgag ttagagcttc gtttattaca gagagtggac
10441 actggtcttg aacatcctat tggcagctct gaatggcttt tgtcagcaca caaacagttg
10501 acccaggata tgtctactca gagggcaatt cagacagaga aagagcagca gatagaaacg
10561 gtctgtgaaa caattcagaa totggttgat aatataaaga ctgtgctcac tggtcataac
10621 cgacagcttg gagatgtcaa acatctctcg aaagctatgg ctaaggatga agaagctgct
10681 ctggcagatg gtgaagatgt tccctatgag aacagtgtta ggcagttttt gggtgaatat
10741 aaatcatggc aagacaacat tcaaacagtt ctatttacat tagtccaggc tatgggtcag
10801 gttcgaagtc aagaacacgt tgaaatgctc caggaaatca ctcccacctt gaaagaactg
10861 aaaacacaaa gtcagagtat ctataataat ttagtgagtt ttgcatcacc cttagtcacc
10921 gatgcaacaa atgaatgttc gagtccaacg tcatctgcta cttatcagcc atccttcgct
10981 gcagcagtcc ggagtaacac tggccagaag actcagcctg atgtcatgtc acagaatgct
11041 agaaagctga tccagaaaaa tcttgctaca tcagctgata ctccaccaag caccgttcca
11101 ggaactggca agagtgttgo ttgtagtcct aaaaaggcag ccagagaccc taaaactggg
11161 aaagcggtgc aagagagaaa ctcctatgca gtgagtgtgt ggaagagagt gaaagccaag
11221 ttagagggcc gagatgttga tccgaatagg aggatgtcag ttgctgaaca ggttgactat
11281 gtcattaagg aagcaactaa tctagataac ttggctcagc tgtatgaagg ttggacagcc
11341 tgggtgtga
(SEQ ID NO: 8)
SMG1 amino acid sequence (GenBank Accession No. NP_055907.3)
        1 msrrapgsrl ssggggggtk yprswndwqp rtdsasadpd nlkysssrdr ggsssyglqp
      61 snsavvsrgr hddtrvhadi qndekggysv nggsgentyg rkslgqelrv nnvtspefts
    121 vqhgsralat kdmrksqers msysdesrls nllrritred drdrrlatvk qlkefiqqpe
    181 nklvlvkqld nilaavhdvl nesskllqel rqegacclgl lcaslsyeae kifkwifskf
    241 sssakdevkl lylcatykal etvgekkafs svmqlvmtsl qsilenvdtp ellckcvkci
    301 llvarcyphi fstnfrdtvd ilvgwhidht qkpsltqqvs gwlqslepfw vadlafsttl
    361 lgqfledmea yaedlshvas gesvdedvpp psvslpklaa llrvfstvvr sigerfspir
    421 gppiteayvt dvlyrvmrcv taangvffsc avltaanfcv gvllgsldps mtihcdmvit
    481 ygldglcncq tcgtdyiisv lnlltliveq intklpssfv eklfipsskl lflryhkeke
    541 vvavahavyq avlslknipv letayklilg emtcalnnll hslqlpeacs eikheafknh
    601 vfnvdnakfv vifdlsaltt ignaknslig mwalsptvfa llsknlmivh sdlavhfpai
    661 qyavlytlys hctrhdhfis sslsssspsl fdgavistvt tatkkhfsii lnllgillkk
    721 dnlnqdtrkl lmtwalcaav lmkksetyap lfslpsfhkf ckgllantlv edvniclqac
    781 sslhalsssl pddllqrcvd vcrvqlvhsg trirqafgkl lksipldvvl snnnhtqiqe
    841 islalrshms kapsntfhpq dfsdvisfil ygnshrtgkd nwlerlfysc qrldkrdqst
    901 iprnllktda vlwqwaiwea aqftvlsklr tplgraqdtf qtiegiirsl aahtlnpdqd
    961 vsgwttadnd eghgnnqlrl vlllqylenl eklmynayeg canaltsppk virtffytnr
  1021 qtcqdwltri rlsimrvgll agqpavtvrh gfdlltemkt tslsqgnele vtimmvveal
  1081 celhcpeaig giavwsssiv gknllwinsv aqgaegrfck asvetqehlc amtgvdccis
  1141 sfdksvltla nagrnsaspk hslngearkt vlskptdssp evinylgnka cecyisiadw
  1201 aavqewqnai hdlkkstsst slnlkadfny ikslssfcsg kfvectcglc llpgeninll
  1261 aggskckidm kkllpnmlsp dprelqksie vqllrssvcl atalnpieqd gkwqsitenv
  1321 vkylkqtsri aigplrlstl tvsqslpvls tlqlycssal entvsnrlst edcliplfse
  1381 alrsckghdv rpwmgalryt mygnglleki kcgtvpirsh lmelgltaak farkrgnvsl
  1441 atrllagcse vglgktttaq dlvghfkkls tqgqvdekwg peldiektkl lytagqstha
  1501 memlsscais fcksvkaeya vaksiltlak wiqaewkeis gqlkqvyraq hqqnftglst
  1561 lskniltlie lpsvntmeee ypriesestv higvgepdfi lgqlyhlssv qapevakswa
  162l alaswayrwg rkvvdnasqg egvrllprek sevqnllpdt iteeekeriy gilgqavcrp
  1681 agiqdeditl qitesednee ddmvdviwrg lisscpwlse ldesategvi kvwrkvvdri
  1741 fslyklscsa yftflklnag qipldeddpr lhlshrveqs tddmiviatl rllrllvkha
  1801 gelrgylehg lettptapwr giipqlfsrl nhpevyvrqs icnllcrvaq dsphli1ypa
  1861 ivgtislssc sgasgnkfst aiptllgniq geellvsece ggsppasqds nkdepksgln
  1921 edqammgdcy skivdklssa nptmvlqvqm lvaelrrvtv lwdelwlgvl lqqhmyvlrr
  1981 iqqledevkr vqnnntlrke ekiaimrckh talmkpivfa lehvrsitaa paetphekwf
  2041 qdnygdaien aleklktpln pakpgsswip fkeimlslgq raqkrasyil rleeispwla
  2101 amtnteialp gevsardtvt ihsvggtiti lptktkpkkl lflgsdgksy pylfkgledl
  2161 hldcrimgfl sivntmfati nrqetprfha rhysvtplgt rsgliqwvdg atpltglykr
  2221 wqqreaalqa gkagdsyqtp qnpgivprps elyyskigpa lktvglsldv srrdwplhvm
  2281 kavleelmea tppnllakel wsscttpdcw wrvtqsyars tavmsmvgyi iglgdrhldn
  2341 vlidmttgev vhidynvcfe kgkslrvpek vpfrmtqnie talgvtgvcg vfrlsceqvl
  2401 himrrgretl ltllcafvyd plvdwtaggc agfagavygg ggqgaeskqs kremereitr
  2461 slfssrvaei kvnwfknrde mlvvlpkldg sldeylslqe qltdvekleg klleeiefle
  2521 gaegvdhpsh tlqhryseht qlqtgqravq eaiqvklnef eqwithyqaa fnnleatqla
  2581 sllqeistqm dlgppsyvpa taflqnagqa hlisqceqle ecvgallqqr rsvlrgcleg
  2641 lhhyatvalq ypkaifgkhr ieqwktwmee licnttverc qelyrkyemq yaqgppptve
  2701 qfitatemtl qryaadinsr lirqvcrlkq eavtvpvced qlkeiercik vflhengeeg
  2761 slslasviis alctltrrnl mmegaassag eqlvdltsrd gawfleelcs msgnvtclvg
  2821 llkqchlvpq dldipnpmea setvhlangv ytslqelnsn frqiifpeal rclmkgeytl
  2881 esmlheldgl ieqttdgvpl qtlveslqay lrnaamglee ethahyidva rllhaqygel
  2941 iqprngsvde tpkmsagqml lvafdgmfaq vetafsllve klnkmeipia wrkidiirea
  3001 rstgvnffdd dnhrgvleei fflkrlqtik effrlcgtfs ktlsgsssle dqntvngpvq
  3061 ivnvktlfrn scfsedqmak pikaftadfv rqlliglpnq algltlosfi salgvdiiaq
  3121 veakdfgaes kvsvddlckk avehnigigk fsglvmnrat vlassydtaw kkhdivrrle
  3181 tsisscktsl qrvqlhiamf qwqhedllin rpqamsvtpp prsailtsmk kklhtlsgie
  3241 tsiatvqwkl aalessieqr lkwagganpa lapvlqdfea tiaerrnlvl kesqrasgvt
  3301 flcsniihfe slrtrtaeal nldaalfeli krcqqmcsfa sqfnssvsel elrllqrvdt
  3361 glehpigsse wllsahkqlt qdmstgraiq tekeqqietv cetiqnlvdn iktvllghnr
  3421 qlgdvkhllk amakdeeaal adgedvpyen svrqflgeyk swqdniqtvl ftlqamgqv
  3481 rsqehvemlq eitptlkelk tqsqsiynnl vsfasplvtd atnecsspts satyqpsfaa
  3541 avrsntgqkt qpdvmsqnar kliqknlats adtppstvpg tgksvacspk kavrdpktgk
  3601 avqernsyav svwkrvkakl egrdvdpnrr msvaeqvdyv ikeatnldnl aqlyegwtaw
  3661 v
(SEQ ID NO: 9)
SMG5 nucleotide sequence (GenBank Accession No. NM_015327.2, nt 150-3200)
    150 a tgagccaagg cccccccaca ggggagagca
    181 gcgagcccga agcaaaagtc ctccacacta agcggcttta ccgggctgtg gtggaggctg
    241 tgcatcgact tgacctcatc ctttgcaaca aaactgctta tcaagaagta ttcaaaccag
    301 aaaacattag cctgaggaac aagctgcgtg agctctgogt caagcttatg ttcctgcacc
    361 cagtggacta tgggagaaag gctgaggagc tgctgtggag aaaggtatac tatgaagtta
    421 tccagcttat caagactaac aaaaagcaca tccacagccg gagcactttg gaatgtgcct
    481 acaggacgca cctggttgct ggtattggct tctaccagca tctccttctc tatatccagt
    541 cccactacca gctggaactg cagtgctgca tcgactggac ccatgtcact gaccccctca
    601 taggatgcaa gaagccagtg tctgcctcag ggaaggagat ggattgggca cagatggcat
    661 gtcaccgatg tctggtgtat ctggaggatt tgtcccgata tcagaatgaa ttagctggcg
    721 tagataccca gctgctagcc gagagatttt actaccaagc cctgtcagta gctcctcaga
    781 ttggaatgcc cttcaatcag ctgggcaccc tggcaggcag caagtactat aatgtggaag
    841 ccatgtattg ctacctgcgc tgcatccagt cagaagtgtc ctttgaggga gcctatggga
    901 acctcaagcg gctgtatgac aaggcagcca aaatgtacca ccaactgaag aagtgtgaga
    961 ctcggaaact gtctcctggc aaaaagcgat gtaaagacat taaaaggttg ctagtgaact
  1021 ttatgtatct gcaaagcctc ctacagccca aaagccaggt cgtggactca gagctgacct
  1081 cactttgcca gtcagtcctg gaggacttca acctctgcct cttctacctg ccctcctcac
  1141 ccaacctcag cctggccagt gaggatgagg aggagtatga gagtggatat gctttcctcc
  1201 cggaccttct catctttcaa atggtcatca tctgccttat gtgtgtgcac agcttggaga
  1261 gagcaggatc caagcagtac agtgcagcca ttgccttcac cctggccctc ttttcccacc
  1321 tcgtcaatca tgtcaacata cggctgcagg ctgagctgga agagggcgag aatcccgtcc
  1381 cggcattcca gagtgatggc acagatgaac cagagtccaa ggaacctgtg gagaaagagg
  1441 aggagccaga tcctgagcct cctcctgtaa caccccaagt gggtgagggc agaaagagcc
  1501 gtaagttctc tcgcctctcc tgtctccgcc gtcgccgcca cccacccaaa gttggtgatg
  1561 acagtgacct gagtgaaggc tttgaatcgg actcaagcca tgactcagcc cgggccagtg
  1621 agggctcaga cagtggctct gacaagagtc ttgaaggtgg gggaacggcc tttgatgctg
  1681 aaacagactc ggaaatgaat agccaggagt cccgatcaga cttggaagat atggaggaag
  1741 aggaggggac acggtcacca accctggagc cccctcgggg cagatcagag gctcccgatt
  1801 ccctcaatgg cccactgggc cccagtgagg ctagcattgc cagcaatcta caagccatgt
  1861 ccacccagat gttccagact aagcgctgct tccgactggc ccccaccttt agcaacctgc
  1921 tcctccagcc caccaccaac cctcatacct cggccagcca caggccttgc gtcaatgggg
  1981 atgtagacaa gccttcagag ccagcctctg aggagggctc tgagtcggag gggagtgagt
  2041 ccagtggacg ctcctgtcgg aatgagcgca gcatccagga gaagcttcag gtcctgatgg
  2101 ccgaaggtct gcttcctgct gtgaaagtct tcctggactg gcttcggacc aaccccgacc
  2161 tcatcatcgt gtgtgcgcag agctctcaaa gtctgtggaa ccgcctgtct gtgttgctga
  2221 atctgttgcc tgctgctggt gaactccagg agtctggcct ggccttgtgt cctgaggtcc
  2281 aagatcttct tgaaggttgt gaactgcctg acctcccctc tagccttctg ctcccagagg
  2341 acatggotct tcgtaacctg cccccgctcc gagctgccca cagacgcttt aactttgaca
  2401 cggatcggcc cctgctcagc accttagagg agtcagtggt gcgcatctgc tgcatccgca
  2461 gctttggtca tttcatcgcc cgcctgcaag gcagcatcct gcagttcaac ccagaggttg
  2521 gcatcttcgt cagcattgcc cagtctgagc aggagagcct gctgcagcag gcccaggcac
  2581 agttccgaat ggcacaggag gaagctcgtc ggaacaggct catgagagac atggctcagc
  2641 tacgacttca gctcgaagtg tctcagctgg agggcagcct gcagcagccc aaggcccagt
  2701 cagccatgtc tccctacctc gtccctgaca cccaggccct ctgccaccat ctccctgtca
  2761 tccgccaact ggccaccagt ggccgcttca ttgtcatcat cccaaggaca gtgatcgatg
  2821 gcctagattt gctgaagaag gaacacccag gggcccggga tgggattcgg tacctggagg
  2881 cagagtttaa aaaaggaaac aggtacattc gctgccagaa agaggtggga aagagctttg
  2941 agcggcataa gctgaagagg caggatgcag atgcctggac tctctataag atcctagaca
  3001 gctgcaaaca gctgactctg gcccaggggg caggtgagga ggatccgagt ggcatggtga
  3061 ccatcatcac aggccttcca ctggacaacc ccagcgtgct ttcaggcccc atgcaggcag
  3121 ccctgcaggc cactgcccac gccagtgtgg acatcaagaa tgttctcgac ttctacaagc
  3181 agtggaagga aattggttga
(SEQ ID NO: 10)
SMG5 amino acid sequence (GenBank Accession No. NP_056142.2)
        1 msqgpptges sepeakvlht krlyravvea vhrldlilcn ktayqevfkp enislrklr
      61 elcvklmflh pvdygrkaee llwrkvyyev iqliktnkkh ihsrstleca yrthlvagig
    121 fyghlllyig shyglelqcc idwthvtdpl igckkpvsas gkemdwaqma chrcivylgd
    181 lsryqnelag vdtellaerf yygalsvapq igmpfnqlgt lagskyynve amycylrciq
    241 sevsfegayg nlkrlydkaa kmyhqlkkce trklspokkr ckdikrllvn fmylqsllqp
    301 ksssvdselt slcqsvledf nlclfylpss pnlslasede eeyesgyafl pdllifqmvi
    361 iclmovhsle ragskqysaa iaftlalfsh lvnhvnirlq aeleegenpv pafqsdgtde
    421 pcskcpvckc eepdpcpppv tpgvgegrks rkfsrlsclr rrrhppkvgd dsdlsegfes
    481 dsshdsaras egsdsgsdks legggtafda etdsemnsqe srsdledmee eegtrsptle
    541 pprgrseapd slngplgpse asiasnlgam stqmfqtkrc frlaptfsnl llqpttnpht
    601 sashrpcvng dvdkpsepas eegsesegse ssgrscrner siqeklqvlm aegllpavkv
    661 fldwlrtnpd liivcaqssq slwnrlsvll nllpaagclq esglalcpev qdllegcelp
    721 dlpsslllpe dmalrnlppl raahrrfnfd tdrpllstle esvvriccir sfghfiarlq
    781 gsilqfnpev gifvsiaqse qesllqqaqa qfrmaqeear rnrlmrdmaq lrlqlevsql
    841 egslqqpkaq samspylvpd tqalchhlpv irqlatsgrf iviiprtvid gldllkkehp
    901 gardgiryle aefkkgnryi rcqkevgksf erhklkrqda dawtlykild sckgltlaqg
    961 ageedpsgmv tiitglpldn psvlsgpmqa alqaaahasv diknvldfyk qwkeig
(SEQ ID NO: 11)
SMG6 nucleotide sequence (GenBank Accession No. BC064916.1, nt 296-1831)
    296 atgga
    301 gacattccct gcagtggctg agaaggtcct caaggagttc caggtgttac tgcagcacag
    361 cccctctccc attggaagta cccgcatgct gcagcttatg accatcaata tgtttgcagt
    421 acacaactcc cagctgaaag actgcttctc ggaggagtgc cgctctgtga tccaggaaca
    481 agccgcagct ctgggcttgg ccatgttttc tctactggtc cgccgctgca cctgcttact
    541 taaggagtcc gccaaagctc agctgtcctc tcctgaggac caggatgacc aagacgacat
    601 caaggtgtct tcctttgtcc cggacctgaa ggagctgctc cccagtgtca aagtctggtc
    661 agattggatg ctcggctacc cggacacctg gaatcctcct cccacatccc tggatctgcc
    721 ctcgcatgtt gctgtggatg tatggtcgac gctggctgat ttctgtaaca tactgactgc
    781 agtgaatcag tctgaggtgc cactgtacaa ggacccggat gatgacctca cccttcttat
    841 cctggaagag gatcggcttc tctcgggctt tgtccccttg ctggctgccc ctcaggaccc
    901 ctgctacgtg gagaaaacct cggataaggt tattgcagct gactgcaaaa gggtcacagt
    961 gctgaagtat tttctggaag ccctttgtgg acaagaagag cctctgctgg cattcaaggg
  1021 tggaaagtat gtgtcagtgg cacccgtccc agacaccatg ggaaaggaaa tgggaagcca
  1081 agagggaaca cgactggaag atgaggagga ggatgtggtg attgaagact ttgaggaaga
  1141 ttcagaggct gaaggcagcg gaggcgagga tgacatcagg gagcttcggg ccaagaagct
  1201 ggctctggcc aggaagatag ctgagcagca gcgtcgccag gaaaagatcc aggctgtcct
  1261 ggaggaccac agtcagatga ggcagatgga gctcgaaatc agacctttgt tcctcgtacc
  1321 agacaccaac ggcttcattg accacctggc cagtctggcg cggctgctgg agagcaggaa
  1381 gtacatcctg gtggtgcccc tcatcgtgat caatgagctg gacggcctgg ccaaggggca
  1441 ggagacagac caccgggctg ggggctacgc ccgtgtggta caagagaagg cccgcaagtc
  1501 catcgagttc ctcgagcagc gattcgagag tcgggactct tgcctgcgag ccctgaccag
  1561 ccgtggcaat gaactogaat ccatcgcctt ccgcagtgag gacatcactg gccagctggg
  1621 taacaacgat gatctcatcc tgtcctgctg cctccactac tgcaaagaca aggctaagga
  1681 cttcatgccc gccagcaaag aggagccaat ccggctactc cgggaggtgg tgctgttgac
  1741 ggatgaccgg aacctgcgtg tgaaggcgct cacaaggaat gttcctgtac gggacatccc
  1801 agccttcctc acgtgggccc aggtgggctg a
(SEQ ID NO: 12)
SMG6 amino acid sequence (GenBank Accession No. AAH64916.1)
        1 metfpavaek vlkefqvllq hspspigstr mlqlmtinmf avhnsqlkdc fseecrsviq
      61 eqaaalglam fsllvrrctc llkcsakagl sspedqddqd dikvssfvpd lkellpsvkv
    121 wsdwmlgypd twnppptsld lpshvavdvw stladfcnil tavnqsevpl ykdpdddltl
    181 lileedrlls gfvpllaapq dpcyvektsd kviaadckrv tvlkyfleal cgqeepllaf
    241 kggkyvsvap vpdtmgkcmg sqegtrlede eedvviedfc edseaegsgg eddirelrak
    301 klalarkiae qqrrqekiqa vledhsqmrq meleirplf1 vpdtngfidh laslarlles
    361 rkyilvvpli vineldglak gqetdhragg yarvvqekar ksiefleqrf esrdsclral
    411 tsrgnelesi afrseditgq lgnnddlils cclhyckdka kdfmpaskee pirllrevvl
    481 ltddrnlrvk altrnvpvrd ipafltwagv g
(SEQ ID NO: 13)
SMG7 nucleotide sequence (GenBank Accession No. BC036381.1, nt 119-3655)
    119 at
    121 gagcctgcag agcgcgcagt acctcoggca ggcagaagtc ctgaaggctg acatgacaga
    181 ttctaagctg ggtccagctg aagtctggac atccaggcag gctctgcagg acctgtacca
    241 gaaaatgcta gttaccgatt tggaatacgc tttagacaag aaagtagaac aggatctctg
    301 gaatcacgcc tttaagaatc agatcacaac actacaaggc caggcaaaga atcgagcaaa
    361 tccgaatcgg agtgaagttc aggcaaacct ttctctgttc ctagaggcag ctagtggctt
    421 ctatactcag ttattacaag aactgtgtac agtatttaat gtagatttac catgccgtgt
    481 gaagtcttcc caattgggaa ttatcagcaa taaacagacg cataccagcg ccatagtgaa
    541 gccacagtct agctcctgtt cctatatctg ccagcactgc ctogtccacc ttgcagacat
    601 tgctcgatac agaaaccaga ccagccaggc agagtcctac tataggcatg cagctcagct
    661 tgtcccctcc aatggtcagc cttataatca gttggctatc ttagcttctt ccaaaggaga
    721 ccatctgacc acaattttct actactgcag aagcattgct gtgaagttcc ctttcccagc
    781 tgcctccact aatctgcaaa aagcactttc taaagcactc gaaagccgag atgaggtgaa
    841 aaccaagtgg ggtgtttctg acttcatcaa ggcctttatt aaattccacg gtcatgtgta
    901 cctgagtaag agcttggaaa agttgagccc tcttcgagac aaattggaag aacagtttaa
    961 gaggctgcta ttccaaaaag ctttcaactc tcagcagtta gttcatgtca ctgtcattaa
  1021 cctgtttcaa cttcatcacc ttcgtgactt tagcaatgaa accgagcagc acacttatag
  1081 ccaagatgag cagctatgtt ggacacagtt gctggccctc tttatgtctt ttctcggcat
  1141 cctgtgcaag tgtcctctac agaatgagtc tcaggaggag tcctacaatg cctatcctct
  1201 tccagcagtc aaggtctcca tggactggct aagactcaga cccagggtct ttcaggaggc
  1261 agtggtggat gaaagacagt acatttggcc ctggttgatt tctcttctga atagtttcca
  1321 tccccatgaa gaggacctct caagtattag tgcgacacca cttccagagg agtttgaatt
  1381 acaaggattt ttggcattga gaccttcttt caggaacttg gatttttoca aaggtcacca
  1441 gggtattaca ggggacaaag aaggccagca acgacgaata cgacagcaac gcttgatctc
  1501 tataggcaaa tggattgctg ataatcagcc aaggctgatt cagtgtgaaa atgaggtagg
  1561 gaaattgttg tttatcacag aaatcccaga attaatactg gaagacccca gtgaagccaa
  1621 agagaacctc attctgcaag aaacatctgt gatagagtcg ctggctgcag atgggagccc
  1681 agggctaaaa tcagtgctat ctacaagccg aaatttaagc aacaactgtg acacaggaga
  1741 gaagccagtg gttaccttca aagaaaacat taagacacga gaagtgaaca gagaccaagg
  1801 aagaagtttt cctcccaaag aggtaaaatc ccagacagaa ctaagaaaga ctccagtgtc
  1861 tgaagccaga aaaacacctg taactcaaac cccaacacaa gcaagtaact cccagttcat
  1921 ccccattcat caccctggag ccttccctcc tcttcccagc aggccagggt ttccgccccc
  1581 aacatatgtt atccccccgc ctgtggcatt ttctatgggc tcaggttaca ccttcccagc
  2041 tggtgtttct gtcccaggaa cctttcttca gcctacagct cactctcaag caggaaacca
  2101 ggtgcaagct gggaaacagt cccacattcc ttacagccag caacggccct ctggaccagg
  2161 gccaatgaac cagggacctc aacaatcaca gccaccttcc cagcaacccc ttacatcttt
  2221 accagctcag ccaacagcac agtctacaag ccagctgcag gttcaagctc taactcagca
  2281 acaacaatcc cctacaaaag ctgtgccggc tttggggaaa agcccgcctc accactctgg
  2341 attccagcag tatcaacagg cagatgcctc caaacagctg tggaatcccc ctcaggttca
  2401 aggcccatta gggaaaatta tgcctgtgaa acagcoctac taccttcaga cccaagaccc
  2461 cataaaactg tttgagccgt cattgcaacc tcctgtaatg cagcagcagc ctctagaaaa
  2521 aaaaatgaag ccttttccca tggagccata taaccataat ccctcagaag tcaaggtccc
  2581 agaattctac tgggattctt cctacagcat ggctgataac agatctgtaa tggcacagca
  2641 agcaaacata gaccgcaggg gcaaacggtc accaggaatc ttccgtccag agcaggatcc
  2701 tgtacccaga atgccgtttg aggaccccaa gagctcccct ctgcttcctc cggacctgtt
  2761 aaagagtctg gctgccttgg aggaagagga agagctgatt ttttctaaca ctcctgatct
  2821 ttacccggct ctgctggggc ctctcgcctc tcttcctgga cgaagccttt ttaaatcctt
  2881 attggagaag ccctcagagc tcatgtcaca ttcatcctct ttcctgtccc tcaccggatt
  2841 ctctctcaat caggaaagat acccaaataa tagtatgttc aatgaggtat atgggaaaaa
  3001 cctgacatcc agctccaaag cagaactcag tccctcaatg gccccccagg aaacatctct
  3061 gtattccctt tttgaaggga ctcogtggto tocatcactt cctgccagtt cagatcattc
  3121 aacaccagcc agccagtctc ctcattccto taacccaagc agcctaccca gctctcctcc
  3181 aacacacaac cataattctg ttccattctc caattttgga cccattggga ctocagataa
  3241 cagggataga aggactgcag atcggtggaa aactgataag ccagccatgg gtgggtttgg
  3301 cattgattat ctctcagcaa cgtcatcctc tgagagcagt tggcatcagg ccagcactcc
  3361 gagtggcacc tggacaggcc atggcccttc catggaggat tcctctgctg tcctcatgga
  3421 aagcctaaag aagcaacagc atggggtcca gcagttgggg cccaaaagac agtctgaaga
  3481 ggaaggaagc agcagtatct gcgtagccca cagagggccc aggcccctgc ccagctgcag
  3541 tctcccagcc tccactttca gagtgaaatt caaggcagca cggacatgtg cccatcaggc
  3601 acagaagaaa acacgacgtc gtccattttg gaagagacga aagaaaggaa aataa
(SEQ ID NO: 14)
SMG7 amino acid sequence (GenBank Accession No. AAH36381.1)
        1 mslgsagylr gaevlkadmt dsklgpaevw tsrqalqdly gkmlvtdley aldkkveqdl
      61 wnhafknqit tlqgqaknra npnrsevqan lslfleaasg fytqllqelc tvfnvdlpcr
    121 vkssqlgiis nkgthtsaiv kpqssscsyi cqhclvhlgd iaryrnqtsq aesyyrhaaq
    181 lvpsngqpyn glailasskg dhlttifyyc rsiavkfpfp aastnlqkal skalesrdev
    241 ktkwgvsdfi kafikfhghv ylskslckls plrekleeqf krllfqkafn sqqlvhvtvi
    301 nlfqlhhlrd fsneteqhty sqdeqlcwtq llalfmsflg ilckcplqnc sqeesynayp
    361 lpavkysmdw lrlrprvfqc avvdergyiw pwlisllnsf hpheedlssi satplpeefe
    421 lggflalrps frnldfskgh qgitgdkcgq qrrirqqrli sigkwiadnq prliqcenev
    481 gkllfiteip eliledpsea kenlilqets vieslaadgs pglksvlsts rnlsnncdtg
    541 ekpvvtfken iktrevnrdg grsfppkcvk sqtelrktpv searktpvtq tptqasnsqf
    601 ipihhpgafp plpsrpgfpp ptyvipppva fsmgsgytfp agvsvpqtfl qptahspagn
    661 qvqagkqshi pysqqrpsgp gpmnqgpqqs qppsqqplts lpaqptaqst sqlqvqaltq
    721 qqqsptkavp algkspphhs gfggyqqada skqlwnpqgv qgplgkimpv kqpyylqtqd
    781 piklfepslq ppvmqqqple kkmkpfpmep ynhnpsevkv pefywdssys madnrsvmaq
    841 qanidrrgka spgifrpeqd pvprmpfedp ksspllppdl lkslaaleee eelif sntpd
    901 lypallgpla slpgrslfks llekpselms hsssflsltg fslnqerypn nsmfnevygk
    961 nltssskael spsmapqcts lyslfegtpw spslpassdh stpasqsphs snpsslpssp
  1021 pthnhnsvpf snfgpigtpd nrdrrtadrw ktdkpamggf gidylsatss sesswhqast
  1081 psgtwtghgp smedssavlm eslkkqqkgv qqlqpkrqse eegsssicva hrgprplpsc
  1141 slpastfrvk fkaartcahq aqkktrrrpf wkrrkkgk
```

We claim:

1. A method of reducing FUS/TLS or TDP-43 toxicity in a neuronal cell or glial cell suffering from or susceptible to such toxicity, comprising:
providing to the cell a therapeutically effective amount of an NMD polypeptide, thereby reducing the FUS/TLS or TDP-43 toxicity in the cell.

2. The method of claim 1, wherein the therapeutically effective amount is an amount that is correlated with a statistically significant probability of reducing FUS/TLS or TDP-43 toxicity in the cell.

3. The method of claim 1, wherein the step of providing comprises administering a composition comprising the NMD polypeptide.

4. The method of claim 1, wherein the NMD polypeptide is a UPF1, UPF2, UPF3, SMG1, SMG5, SMG6, or SMG7 polypeptide.

5. The method of claim 1, wherein the step of providing comprises administering a composition comprising a nucleic acid encoding the NMD polypeptide.

6. The method of claim 5, wherein the nucleic acid encodes a UPF1, UPF2, UPF3, SMG1, SMG5, SMG6, or SMG7 polypeptide.

7. The method of claim 1, wherein the step of providing comprises administering a composition comprising an activator of the NMD polypeptide.

8. The method of claim 1, wherein the NMD polypeptide is provided in vitro.

9. The method of claim 1, wherein the NMD polypeptide is provided in vivo.

10. The method of claim 1, wherein the cell is a human neuronal cell or a human glial cell.

11. The method of claim 1, wherein the NMD polypeptide comprises the amino acid sequence of SEQ ID NO:2, 4, 6, 8, 10, 12, or 14, or comprises an amino acid sequence at least 70%, 75%, 80%, 85%, 90%, 91%, 92%, 93%, 94%, 95%, 96%, 97%, 98%, or 99% identical to the amino acid sequence of SEQ ID NO:2, 4, 6, 8, 10, 12, or 14.

12. The method of claim 1, wherein the nucleic acid comprises the nucleic acid sequence of SEQ ID NO:1, 3, 5, 7, 9, 11, or 13, or comprises a nucleic acid sequence at least 70%, 75%, 80%, 85%, 90%, 91%, 92%, 93%, 94%, 95%, 96%, 97%, 98%, or 99% identical to the nucleic acid sequence of SEQ ID NO:1, 3, 5, 7, 9, 11, or 13.

13. The method of claim 1, wherein the UPF1 polypeptide comprises the amino acid sequence of SEQ ID NO:2, or comprises an amino acid sequence at least 70%, 75%, 80%, 85%, 90%, 91%, 92%, 93%, 94%, 95%, 96%, 97%, 98%, or 99% identical to the amino acid sequence of SEQ ID NO:2.


SEQUENCE LISTING (14 sequences)

SEQ ID NO: 1 - DNA, 3357 bp, Homo sapiens
  source 1..3357: mol_type = genomic DNA
atgagcgtggaggcgtacgggcccagctcgcagactctcactttcctggacacggaggaggccgagctgcttggcgccgacacacagggctccgagttcgagttcaccgactttactcttcctagccagacgcagacgccccccggcggccccggcggcccgggcggtggcggcgcgggaagcccgggcggcgcgggcgccggcgctgcggcgggacagctcgacgcgcaggttgggcccgaaggcatcctgcagaacggggctgtggacgacagtgtagccaagaccagccagttgttggctgagttgaacttcgaggaagatgaagaagacacctattacacgaaggacctccccatacacgcctgcagttactgtggaatacacgatcctgcctgcgtggtttactgtaataccagcaagaagtggttctgcaacggacgtggaaatacttctggcagccacattgtaaatcaccttgtgagggcaaaatgcaaagaggtgaccctgcacaaggacgggcccctgggggagacagtcctggagtgctacaactgcggctgtcgcaacgtcttcctcctcggcttcatcccggccaaagctgactcagtggtggtgctgctgtgcaggcagccctgtgccagccagagcagcctcaaggacatcaactgggacagctcgcagtggcagccgctgatccaggaccgctgcttcctgtcctggctggtcaagatcccctccgagcaggagcagctgcgggcacgccagatcacggcacagcagatcaacaagctggaggagctgtggaaggaaaacccttctgccacgctggaggacctggagaagccgggggtggacgaggagccgcagcatgtcctcctgcggtacgaggacgcctaccagtaccagaacatattcgggcccctggtcaagctggaggccgactacgacaagaagctgaaggagtcccagactcaagataacatcactgtcaggtgggacctgggccttaacaagaagagaatcgcctacttcactttgcccaagactgactctgacatgcggctcatgcagggggatgagatatgcctgcggtacaaaggggaccttgcgcccctgtggaaagggatcggccacgtcatcaaggtccctgataattatggcgatgagatcgccattgagctgcggagcagcgtgggtgcacctgtggaggtgactcacaacttccaggtggattttgtgtggaagtcgacctcctttgacaggatgcagagcgcattgaaaacgtttgccgtggatgagacctcggtgtctggctacatctaccacaagctgttgggccacgaggtggaggacgtaatcaccaagtgccagctgcccaagcgcttcacggcgcagggcctccccgacctcaaccactcccaggtttatgccgtgaagactgtgctgcaaagaccactgagcctgatccagggcccgccaggcacggggaagacggtgacgtcggccaccatcgtctaccacctggcccggcaaggcaacgggccggtgctggtgtgtgctccgagcaacatcgccgtggaccagctaacggagaagatccaccagacggggctaaaggtcgtgcgcctctgcgccaagagccgtgaggccatcgactccccggtgtcttttctggccctgcacaaccagatcaggaacatggacagcatgcctgagctgcagaagctgcagcagctgaaagacgagactggggagctgtcgtctgccgacgagaagcggtaccgggccttgaagcgcaccgcagagagagagctgctgatgaacgcagatgtcatctgctgcacatgtgtgggcgccggtgacccgaggctggccaagatgcagttccgctccattttaatcgacgaaagcacccaggccaccgagccggagtgcatggttcccgtggtcctcggggccaagcagctgatccttgtaggcgaccactgccagctgggcccagtggtgatgtgcaagaaggcggccaaggccgggctgtcacagtcgctcttcgagcgcctggtggtgctgggcatccggcccatccgcctgcaggtccagtaccggatgcaccctgcactcagcgccttcccatccaacatcttctacgagggctccctccagaatggtgtcactgcagcggatcgtgtgaagaagggatttgacttccagtggccccaacccgataaaccgatgttcttctacgtgacccagggccaagaggagattgccagctcgggcacctcctacctgaacaggaccgaggctgcgaacgtggagaagatcaccacgaagttgctgaaggcaggcgccaagccggaccagattggcatcatcacgccctacgagggccagcgctcctacctggtgcagtacatgcagttcagcggctccctgcacaccaagctctaccaggaagtggagatcgccagtgtggacgcctttcagggacgcgagaaggacttcatcatcctgtcctgtgtgcgggccaacgagcaccaaggcattggctttttaaatgaccccaggcgtctgaacgtggccctgaccagagcaaggtatggcgtcatcattgtgggcaacccgaaggcactatcaaagcagccgctctggaaccacctgctgaactactataaggagcagaaggtgctggtggaggggccgctcaacaacctgcgtgagagcctcatgcagttcagcaagccacggaagctggtcaacactatcaacccgggagcccgcttcatgaccacagccatgtatgatgcccgggaggccatcatcccaggctccgtctatgatcggagcagccagggccggccttccagcatgtacttccagacccatgaccagattggcatgatcagtgccggccctagccacgtggctgccatgaacattcccatccccttcaacctggtcatgccacccatgccaccgcctggctattttggacaagccaacgggcctgctgcagggcgaggcaccccgaaaggcaagactggtcgtgggggacgccagaagaaccgctttgggcttcctggacccagccagactaacctccccaacagccaagccagccaggatgtggcgtcacagcccttctctcagggcgccctgacgcagggctacatctccatgagccagccttcccagatgagccagcccggcctctcccagccggagctgtcccaggacagttaccttggtgacgagtttaaatcacaaatcgacgtggcgctctcacaggactccacgtaccagggagagcgggcttaccagcatggcggggtgacggggctgtcccagtattaa

SEQ ID NO: 2 - protein, 1118 aa, Homo sapiens [sequence omitted]

SEQ ID NO: 3 - DNA, 3819 bp, Homo sapiens
  source 1..3819: mol_type = genomic DNA
atgccagctgagcgtaaaaagccagcaagtatggaagaaaaagactctttaccaaacaacaaggaaaaagactgcagtgaaaggcggacagtgagcagcaaggagaggccaaaagacgatatcaagctcactgccaagaaggaggtcagcaaggcccctgaagacaagaagaagagactggaagatgataagagaaaaaaggaagacaaggaacgcaagaaaaaagacgaagaaaaggtgaaggcagaggaagaatcaaagaaaaaagaagaggaagaaaaaaagaaacatcaagaggaagagagaaagaagcaagaagagcaggccaaacgtcagcaagaagaagaagcagctgctcagatgaaagaaaaagaagaatccattcagcttcatcaggaagcttgggaacgacatcatttaagaaaggaacttcgtagcaaaaaccaaaatgctccggacagccgaccagaggaaaacttcttcagccgcctcgactcaagtttgaagaaaaatactgcttttgtcaagaaactaaaaactattacagaacaacagagagactccttgtcccatgattttaatggcctaaatttaagcaaatacattgcagaagctgtagcttccatcgtggaagcaaaactaaaaatctctgatgtgaactgtgctgtgcacctctgctctctctttcaccagcgttatgctgactttgccccatcacttcttcaggtctggaaaaaacattttgaagcaaggaaagaggagaaaacacctaacatcaccaagttaagaactgatttgcgttttattgcagaattgacaatagttgggattttcactgacaaggaaggtctttccttaatctatgaacagctaaaaaatattattaatgctgatcgggagtcccacactcatgtctctgtagtgattagtttctgtcgacattgtggagatgatattgctggacttgtaccaaggaaagtaaagagtgctgcagagaagtttaatttgagttttcctcctagtgagataattagtccagagaaacaacagcccttccagaatcttttaaaagagtactttacgtctttgaccaaacacctgaaaagggaccacagggagctccagaatactgagagacaaaacaggcgcattctacattctaaaggggagctcagtgaagatagacataaacagtatgaggaatttgctatgtcttaccagaagctgctggcaaattctcaatccttagcagaccttttggatgaaaatatgccagatcttcctcaagacaaacccacaccagaagaacatgggcctggaattgatatattcacacctggtaaacctggagaatatgacttggaaggtggtatatgggaagatgaagatgctcggaatttttatgagaacctcattgatttgaaggcttttgtcccagccatcttgtttaaagacaatgaaaaaagttgtcagaataaagagtccaacaaagatgataccaaagaggcaaaagaatctaaggagaataaggaggtatcaagtcccgatgatttggaacttgagttggagaatctagaaattaatgatgacaccttagaattagagggtggagatgaagctgaagatcttacaaagaaacttcttgatgaacaagaacaagaagatgaggaagccagcactggatctcatctcaagctcatagtagatgctttcctacagcagttacccaactgtgtcaaccgagatctgatagacaaggcagcaatggatttttgcatgaacatgaacacaaaagcaaacaggaagaagttggtacgggcactcttcatagttcctagacaaaggttggatttgctaccattttatgcaagattggttgctacattgcatccctgcatgtctgatgtagcagaggatctttgttccatgctgaggggggatttcagatttcatgtacggaaaaaggaccagatcaatattgaaacaaagaataaaactgttcgttttataggagaactaactaagtttaagatgttcaccaaaaatgacacactgcattgtttaaagatgcttctgtcagacttctctcatcaccatattgaaatggcatgcaccctgctggagacatgtggacggtttcttttcagatctccagaatctcacctgaggaccagtgtacttttggagcaaatgatgagaaagaagcaagcaatgcatcttgatgcgagatacgtcacaatggtagagaatgcatattactactgcaacccacctccagctgaaaaaaccgtgaaaaagaaacgtcctcctctccaggaatatgtccggaaacttttgtacaaagatctctctaaggttaccaccgagaaggttttgagacagatgcgaaagctgccctggcaggaccaagaagtgaaagactatgttatttgttgtatgataaacatctggaatgtgaaatataatagtattcattgtgtagccaacctcttagcaggactagtgctctaccaagaggatgttgggatccacgttgtggatggagtgttagaagatattcgattaggaatggaggttaatcaacctaaatttaatcagaggcgcatcagcagtgccaagttcttaggagaactttacaattaccgaatggtggaatcagctgttattttcagaactctgtattcttttacctcatttggtgttaatcctgatggctctccaagttccctggacccacctgagcatcttttcagaattagactcgtatgcactattctggacacatgtggccagtactttgacagaggttccagtaaacgaaaacttgattgtttccttgtatattttcagcgttatgtttggtggaagaaaagtttggaggtttggacaaaagaccatccatttcctattgatatagattacatgatcagtgatacactagaactgctaagaccaaagatcaaactctgtaattctctggaagaatccatcaggcaggtacaagacttggaacgagaattcttaataaaactaggcctagtaaatgacaaagactcaaaagattctatgacagaaggagaaaatcttgaagaggatgaagaagaagaagaaggtggggctgaaacagaagaacaatctggaaatgaaagtgaagtaaatgagccagaagaagaggagggttctgataatgatgatgatgagggagaagaagaggaggaagagaatacagattaccttacagattccaataaggaaaatgaaaccgatgaagagaatactgaggtaatgattaaaggcggtggacttaagcatgtaccttgtgtagaagatgaggacttcattcaagctctggataaaatgatgctagaaaatctacagcaacgaagtggtgaatctgttaaagtgcaccaactagatgtggccattcctttgcatctcaaaagccagctgaggaaagggcccccactgggaggtggggaaggagaggctgagtctgcagacacaatgccgtttgtcatgttaacaagaaaaggcaataaacagcagtttaagatccttaatgtacccatgtcctctcaacttgctgcaaatcactggaaccagcaacaggcagaacaagaagagaggatgagaatgaagaagctcacactagatatcaatgaacggcaagaacaagaagattatcaagaaatgttgcagtctcttgcacagcgcccagctccagcaaacaccaatcgtgagaggcggcctcgctaccaacatccgaagggagcacctaatgcagatctaatctttaagactggtgggaggagacgttga

SEQ ID NO: 4 - protein, 1272 aa, Homo sapiens
MPAERKKPASMEEKDSLPNNKEKDCSERRTVSSKERPKDDIKLTAKKEVSKAPEDKKKRLEDDKRKKEDKERKKKDEEKVKAEEESKKKEEEEKKKHQEEERKKQEEQAKRQQEEEAAAQMKEKEESIQLHQEAWERHHLRKELRSKNQNAPDSRPEENFFSRLDSSLKKNTAFVKKLKTITEQQRDSLSHDFNGLNLSKYIAEAVASIVEAKLKISDVNCAVHLCSLFHQRYADFAPSLLQVWKKHFEARKEEKTPNITKLRTDLRFIAELTIVGIFTDKEGLSLIYEQLKNIINADRESHTHVSVVISFCRHCGDDIAGLVPRKVKSAAEKFNLSFPPSEIISPEKQQPFQNLLKEYFTSLTKHLKRDHRELQNTERQNRRILHSKGELSEDRHKQYEEFAMSYQKLLANSQSLADLLDENMPDLPQDKPTPEEHGPGIDIFTPGKPGEYDLEGGIWEDEDARNFYENLIDLKAFVPAILFKDNEKSCQNKESNKDDTKEAKESKENKEVSSPDDLELELENLEINDDTLELEGGDEAEDLTKKLLDEQEQEDEEASTGSHLKLIVDAFLQQLPNCVNRDLIDKAAMDFCMNMNTKANRKKLVRALFIVPRQRLDLLPFYARLVATLHPCMSDVAEDLCSMLRGDFRFHVRKKDQINIETKNKTVRFIGELTKFKMFTKNDTLHCLKMLLSDFSHHHIEMACTLLETCGRFLFRSPESHLRTSVLLEQMMRKKQAMHLDARYVTMVENAYYYCNPPPAEKTVKKKRPPLQEYVRKLLYKDLSKVTTEKVLRQMRKLPWQDQEVKDYVICCMINIWNVKYNSIHCVANLLAGLVLYQEDVGIHVVDGVLEDIRLGMEVNQPKFNQRRISSAKFLGELYNYRMVESAVIFRTLYSFTSFGVNPDGSPSSLDPPEHLFRIRLVCTILDTCGQYFDRGSSKRKLDCFLVYFQRYVWWKKSLEVWTKDHPFPIDIDYMISDTLELLRPKIKLCNSLEESIRQVQDLEREFLIKLGLVNDKDSKDSMTEGENLEEDEEEEEGGAETEEQSGNESEVNEPEEEEGSDNDDDEGEEEEEENTDYLTDSNKENETDEENTEVMIKGGGLKHVPCVEDEDFIQALDKMMLENLQQRSGESVKVHQLDVAIPLHLKSQLRKGPPLGGGEGEAESADTMPFVMLTRKGNKQQFKILNVPMSSQLAANHWNQQQAEQEERMRMKKLTLDINERQEQEDYQEMLQSLAQRPAPANTNRERRPRYQHPKGAPNADLIFKTGGRRR

SEQ ID NO: 5 - DNA, 1359 bp, Homo sapiens
  source 1..1359: mol_type = genomic DNA
atgctgtcggccctagaagtgcagttccaccgcgactcgcagcagcaggaggctgagacgccgccaacttcgtcctccggttgcgggggcggtgcgggcaaacctcgcgaggagaagaggacggccctgagcaaggtggtcatccgccgcctgcctccgggcctcaccaaggagcagctggaggagcagctgcgcccgctgccagcacacgactacttcgagttcttcgccgccgacctgagtctttatcctcatctctactcaagagcatacattaattttaggaatcctgatgacatccttctttttagagatcgttttgatggatatatcttccttgacagcaaaggcctagaatatcctgcagtggtagagtttgctccattccagaagatagccaaaaagaagctgagaaaaaaagatgccaagactggaagcatcgaagatgatccagaatataagaagtttttagaaacctactgtgtggaggaagagaagaccagtgccaaccctgagactctgctgggggagatggaggcgaagacaagagagctcattgctagaagaaccacacctcttttggaatatattaaaaatagaaaattagaaaagcagagaattcgagaagagaagcgagaagaacggaggaggagagagttagaaaagaaacgtttgcgggaagaggaaaaaagaagaagaagagaagaagaaagatgcaaaaaaaaagagacagataaacagaagaaaattgcagagaaagaagtaaggattaagcttcttaagaaaccagaaaagggagaggaaccaaccacagagaaaccaaaagaaagaggagaggagattgatactggaggtggcaagcaggaatcctgtgcccccggtgcagtcgtaaaagccaggcccatggaaggctcgctggaggagccccaggagacgtcacacagcggcagtgataaagagcacagggatgtggagagatctcaagaacaagaatctgaagcacaaagataccatgtggatgacggcaggaggcacagagctcaccacgagcctgaacggctttccagaaggagtgaggatgagcagagatgggggaaaggacctggccaagacagagggaagaaggggagccaggacagcggggctccgggggaggccatggagagactgggaagagcgcaaaggtgtgacgacagtccagcacccagaaaagagcgactggcaaacaaggaccggccagccttgcagctgtatgatccaggagctcgcttccgagcgcgagagtgtggcggaaacaggaggatctgcaaggcagaaggttcggggactggtcctgagaagagggaagaggcagagtga

SEQ ID NO: 6 - protein, 452 aa, Homo sapiens [sequence omitted]

SEQ ID NO: 7 - DNA, 10986 bp, Homo sapiens
  source 1..10986: mol_type = genomic DNA
atgagccgcagagccccggggtctcggctgagcagcggcggcggcggcggcggcaccaagtatccgcggagctggaatgactggcaacccagaactgatagtgcatcagccgacccagataatttaaaatattcttcatccagagatagaggtggttcttcctcttatggactgcaaccttcaaattcagctgtggtgtctcggcaaaggcacgatgataccagagtccacgctgacatacagaatgacgaaaagggtggctacagtgtcaatggaggatctggggaaaatacttatggtcggaagtcgttggggcaagagctgagggttaacaatgtgaccagccctgagttcaccagtgttcagcatggcagtcgtgctttagccaccaaagacatgaggaaatcacaggagagatcgatgtcttattctgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttatatttgacctcagtgccctgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttcttcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaagaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagtgctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcagttagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcatgctggtgagcttcggcagtatctggagcacggcttggagacaacacccactgcaccatggagaggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaagcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcaacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtga

SEQ ID NO: 8 - protein, 3661 aa, Homo sapiens
MSRRAPGSRLSSGGGGGGTKYPRSWNDWQPRTDSASADPDNLKYSSSRDRGGSSSYGLQPSNSAVVSRQRHDDTRVHADIQNDEKGGYSVNGGSGENTYGRKSLGQELRVNNVTSPEFTSVQHGSRALATKDMRKSQERSMSYSDESRLSNLLRRITREDDRDRRLATVKQLKEFIQQPENKLVLVKQLDNILAAVHDVLNESSKLLQELRQEGACCLGLLCASLSYEAEKIFKWIFSKFSSSAKDEVKLLYLCATYKALETVGEKKAFSSVMQLVMTSLQSILENVDTPELLCKCVKCILLVARCYPHIFSTNFRDTVDILVGWHIDHTQKPSLTQQVSGWLQSLEPFWVADLAFSTTLLGQFLEDMEAYAEDLSHVASGESVDEDVPPPSVSLPKLAALLRVFSTVVRSIGERFSPIRGPPITEAYVTDVLYRVMRCVTAANQVFFSEAVLTAANECVGVLLGSLDPSMTIHCDMVITYGLDQLENCQTCGTDYIISVLNLLTLIVEQINTKLPSSFVEKLFIPSSKLLFLRYHKEKEVVAVAHAVYQAVLSLKNIPVLETAYKLILGEMTCALNNLLHSLQLPEACSEIKHEAFKNHVFNVDNAKFVVIFDLSALTTIGNAKNSLIGMWALSPTVFALLSKNLMIVHSDLAVHFPAIQYAVLYTLYSHCTRHDHFISSSLSSSSPSLFDGAVISTVTTATKKHFSIILNLLGILLKKDNLNQDTRKLLMTWALEAAVLMKKSETYAPLFSLPSFHKFCKGLLANTLVEDVNICLQACSSLHALSSSLPDDLLQRCVDVCRVQLVHSGTRIRQAFGKLLKSIPLDVVLSNNNHTEIQEISLALRSHMSKAPSNTFHPQDFSDVISFILYGNSHRTGKDNWLERLFYSCQRLDKRDQSTIPRNLLKTDAVLWQWAIWEAAQFTVLSKLRTPLGRAQDTFQTIEGIIRSLAAHTLNPDQDVSQWTTADNDEGHGNNQLRLVLLLQYLENLEKLMYNAYEGCANALTSPPKVIRTFFYTNRQTCQDWLTRIRLSIMRVGLLAGQPAVTVRHGFDLLTEMKTTSLSQGNELEVTIMMVVEALCELHCPEAIQGIAVWSSSIVGKNLLWINSVAQQAEGRFEKASVEYQEHLCAMTGVDCCISSFDKSVLTLANAGRNSASPKHSLNGESRKTVLSKPTDSSPEVINYLGNKACECYISIADWAAVQEWQNAIHDLKKSTSSTSLNLKADFNYIKSLSSFESGKFVECTEQLELLPGENINLLAGGSKEKIDMKKLLPNMLSPDPRELQKSIEVQLLRSSVCLATALNPIEQDQKWQSITENVVKYLKQTSRIAIGPLRLSTLTVSQSLPVLSTLQLYCSSALENTVSNRLSTEDCLIPLFSEALRSCKQHDVRPWMQALRYTMYQNQLLEKIKEQTVPIRSHLMELGLTAAKFARKRGNVSLATRLLAQCSEVQLGKTTTAQDLVQHFKKLSTQGQVDEKWGPELDIEKTKLLYTAGQSTHAMEMLSSCAISFCKSVKAEYAVAKSILTLAKWIQAEWKEISGQLKQVYRAQHQQNFTGLSTLSKNILTLIELPSVNTMEEEYPRIESESTVHIGVGEPDFILGQLYHLSSVQAPEVAKSWAALASWAYRWGRKVVDNASQGEGVRLLPREKSEVQNLLPDTITEEEKERIYGILGQAVCRPAGIQDEDITLQITESEDNEEDDMVDVIWRQLISSCPWLSELDESATEGVIKVWRKVVDRIFSLYKLSCSAYFTFLKLNAGQIPLDEDDPRLHLSHRVEQSTDDMIVMATLRLLRLLVKHAGELRQYLEHGLETTPTAPWRGIIPQLFSRLNHPEVYVRQSICNLLCRVAQDSPHLILYPAIVGTISLSSESQASGNKFSTAIPTLLGNIQGEELLVSECEGGSPPASQDSNKDEPKSGLNEDQAMMQDCYSKIVDKLSSANPTMVLQVQMLVAELRRVTVLWDELWLGVLLQQHMYVLRRIQQLEDEVKRVQNNNTLRKEEKIAIMREKHTALMKPIVFALEHVRSITAAPAETPHEKWFQDNYGDAIENALEKLKTPLNPAKPGSSWIPFKEIMLSLQQRAQKRASYILRLEEISPWLAAMTNTEIALPGEVSARDTVTIHSVGGTITILPTKTKPKKLLFLGSDGKSYPYLFKGLEDLHLDERIMQFLSIVNTMFATINRQETPRFHARHYSVTPLGTRSGLIQWVDGATPLFGLYKRWQQREAALQAQKAQDSYQTPQNPGIVPRPSELYYSKIGPALKTVGLSLDVSRRDWPLHVMKAVLEELMEATPPNLLAKELWSSCTTPDEWWRVTQSYARSTAVMSMVGYIIGLGDRHLDNVLIDMTTGEVVHIDYNVCFEKGKSLRVPEKVPFRMTQNIETALGVTGVEGVFRLSCEQVLHIMRRGRETLLTLLEAFVYDPLVDWTAGGEAGFAGAVYGGGGQQAESKQSKREMEREITRSLFSSRVAEIKVNWFKNRDEMLVVLPKLDGSLDEYLSLQEQLTDVEKLQGKLLEEIEFLEGAEGVDHPSHTLQHRYSEHTQLQTQQRAVQEAIQVKLNEFEQWITHYQAAFNNLEATQLASLLQEISTQMDLGPPSYVPATAFLQNAGQAHLISQCEQLEGEVGALLQQRRSVLRGCLEQLHHYATVALQYPKAIFQKHRIEQWKTWMEELICNTTVERCQELYRKYEMQYAPQPPPTVCQFITATEMTLQRYAADINSRLIRQVERLKQEAVTVPVCEDQLKEIERCIKVFLHENGEEGSLSLASVIISALCTLTRRNLMMEGAASSAGEQLVDLTSRDGAWFLEELCSMSGNVTCLVQLLKQCHLVPQDLDIPNPMEASETVHLANGVYTSLQELNSNFRQIIFPEALRCLMKGEYTLESMLHELDGLIEQTTDGVPLQTLVESLQAYLRNAAMGLEEETHAHYIDVARLLHAQYGELIQPRNGSVDETPKMSAGQMLLVAFDGMFAQVETAFSLLVEKLNKMEIPIAWRKIDIIREARSTQVNFFDDDNHRQVLEEIFFLKRLQTIKEFFRLCGTFSKTLSGSSSLEDQNTVNGPVQIVNVKTLFRNSCFSEDQMAKPIKAFTADFVRQLLIGLPNQALGLTLCSFISALGVDIIAQVEAKDFGAESKVSVDDLCKKAVEHNIQIGKFSQLVMNRATVLASSYDTAWKKHDLVRRLETSISSCKTSLQRVQLHIAMFQWQHEDLLINRPQAMSVTPPPRSAILTSMKKKLHTLSQIETSIATVQEKLAALESSIEQRLKWAGGANPALAPVLQDFEATIAERRNLVLKESQRASQVTFLCSNIIHFESLRTRTAEALNLDAALFELIKRCQQMCSFASQFNSSVSELELRLLQRVDTGLEHPIGSSEWLLSAHKQLTQDMSTQRAIQTEKEQQIETVCETIQNLVDNIKTVLTGHNRQLGDVKHLLKAMAKDEEAALADGEDVPYENSVRQFLGEYKSWQDNIQTVLFTLVQAMGQVRSQEHVEMLQEITPTLKELKTQSQSIYNNLVSFASPLVTDATNECSSPTSSATYQPSFAAAVRSNTGQKTQPDVMSQNARKLIQKNLATSADTPPSTVPGTGKSVACSPKKAVRDPKTGKAVQERNSYAVSVWKRVKAKLEGRDVDPNRRMSVAEQVDYVIKEATNLDNLAQLYEGWTAWV

SEQ ID NO: 9 - DNA, 3051 bp, Homo sapiens
  source 1..3051: mol_type = genomic DNA
atgagccaaggcccccccacaggggagagcagcgagcccgaagcaaaagtcctccacactaagcggctttaccgggctgtggtggaggctgtgcatcgacttgacctcatcctttgcaacaaaactgcttatcaagaagtattcaaaccagaaaacattagcctgaggaacaagctgcgtgagctctgcgtcaagcttatgttcctgcacccagtggactatgggagaaaggctgaggagctgctgtggagaaaggtatactatgaagttatccagcttatcaagactaacaaaaagcacatccacagccggagcactttggaatgtgcctacaggacgcacctggttgctggtattggcttctaccagcatctccttctctatatccagtcccactaccagctggaactgcagtgctgcatcgactggacccatgtcactgaccccctcataggatgcaagaagccagtgtctgcctcagggaaggagatggattgggcacagatggcatgtcaccgatgtctggtgtatctgggggatttgtcccgatatcagaatgaattagctggcgtagataccgagctgctagccgagagattttactaccaagccctgtcagtagctcctcagattggaatgcccttcaatcagctgggcaccctggcaggcagcaagtactataatgtggaagccatgtattgctacctgcgctgcatccagtcagaagtgtcctttgagggagcctatgggaacctcaagcggctgtatgacaaggcagccaaaatgtaccaccaactgaagaagtgtgagactcggaaactgtctcctggcaaaaagcgatgtaaagacattaaaaggttgctagtgaactttatgtatctgcaaagcctcctacagcccaaaagcagctccgtggactcagagctgacctcactttgccagtcagtcctggaggacttcaacctctgcctcttctacctgccctcctcacccaacctcagcctggccagtgaggatgaggaggagtatgagagtggatatgctttcctcccggaccttctcatctttcaaatggtcatcatctgccttatgtgtgtgcacagcttggagagagcaggatccaagcagtacagtgcagccattgccttcaccctggccctcttttcccacctcgtcaatcatgtcaacatacggctgcaggctgagctggaagagggcgagaatcccgtcccggcattccagagtgatggcacagatgaaccagagtccaaggaacctgtggagaaagaggaggagccagatcctgagcctcctcctgtaacaccccaagtgggtgagggcagaaagagccgtaagttctctcgcctctcctgtctccgccgtcgccgccacccacccaaagttggtgatgacagtgacctgagtgaaggctttgaatcggactcaagccatgactcagcccgggccagtgagggctcagacagtggctctgacaagagtcttgaaggtgggggaacggcctttgatgctgaaacagactcggaaatgaatagccaggagtcccgatcagacttggaagatatggaggaagaggaggggacacggtcaccaaccctggagccccctcggggcagatcagaggctcccgattccctcaatggcccactgggccccagtgaggctagcattgccagcaatctacaagccatgtccacccagatgttccagactaagcgctgcttccgactggcccccacctttagcaacctgctcctccagcccaccaccaaccctcatacctcggccagccacaggccttgcgtcaatggggatgtagacaagccttcagagccagcctctgaggagggctctgagtcggaggggagtgagtccagtggacgctcctgtcggaatgagcgcagcatccaggagaagcttcaggtcctgatggccgaaggtctgcttcctgctgtgaaagtcttcctggactggcttcggaccaaccccgacctcatcatcgtgtgtgcgcagagctctcaaagtctgtggaaccgcctgtctgtgttgctgaatctgttgcctgctgctggtgaactccaggagtctggcctggccttgtgtcctgaggtccaagatcttcttgaaggttgtgaactgcctgacctcccctctagccttctgctcccagaggacatggctcttcgtaacctgcccccgctccgagctgcccacagacgctttaactttgacacggatcggcccctgctcagcaccttagaggagtcagtggtgcgcatctgctgcatccgcagctttggtcatttcatcgcccgcctgcaaggcagcatcctgcagttcaacccagaggttggcatcttcgtcagcattgcccagtctgagcaggagagcctgctgcagcaggcccaggcacagttccgaatggcacaggaggaagctcgtcggaacaggctcatgagagacatggctcagctacgacttcagctcgaagtgtctcagctggagggcagcctgcagcagcccaaggcccagtcagccatgtctccctacctcgtccctgacacccaggccctctgccaccatctccctgtcatccgccaactggccaccagtggccgcttcattgtcatcatcccaaggacagtgatcgatggcctggatttgctgaagaaggaacacccaggggcccgggatgggattcggtacctggaggcagagtttaaaaaaggaaacaggtacattcgctgccagaaagaggtgggaaagagctttgagcggcataagctgaagaggcaggatgcagatgcctggactctctataagatcctagacagctgcaaacagctgactctggcccagggggcaggtgaggaggatccgagtggcatggtgaccatcatcacaggccttccactggacaaccccagcgtgctttcaggccccatgcaggcagccctgcaggccgctgcccacgccagtgtggacatcaagaatgttctggacttctacaagcagtggaaggaaattggttga

SEQ ID NO: 10 - protein, 1016 aa, Homo sapiens
MSQGPPTGESSEPEAKVLHTKRLYRAVVEAVHRLDLILCNKTAYQEVFKPENISLRNKLRELCVKLMFLHPVDYGRKAEELLWRKVYYEVIQLIKTNKKHIHSRSTLECAYRTHLVAGIGFYQHLLLYIQSHYQLELQCCIDWTHVTDPLIGCKKPVSASGKEMDWAQMACHRCLVYLGDLSRYQNELAGVDTELLAERFYYQALSVAPQIGMPFNQLGTLAGSKYYNVEAMYCYLRCIQSEVSFEGAYGNLKRLYDKAAKMYHQLKKCETRKLSPGKKRCKDIKRLLVNFMYLQSLLQPKSSSVDSELTSLCQSVLEDFNLCLFYLPSSPNLSLASEDEEEYESGYAFLPDLLIFQMVIICLMCVHSLERAGSKQYSAAIAFTLALFSHLVNHVNIRLQAELEEGENPVPAFQSDGTDEPESKEPVEKEEEPDPEPPPVTPQVGEGRKSRKFSRLSCLRRRRHPPKVGDDSDLSEGFESDSSHDSARASEGSDSGSDKSLEGGGTAFDAETDSEMNSQESRSDLEDMEEEEGTRSPTLEPPRGRSEAPDSLNGPLGPSEASIASNLQAMSTQMFQTKRCFRLAPTFSNLLLQPTTNPHTSASHRPCVNGDVDKPSEPASEEGSESEGSESSGRSCRNERSIQEKLQVLMAEGLLPAVKVFLDWLRTNPDLIIVCAQSSQSLWNRLSVLLNLLPAAGELQESGLALCPEVQDLLEGCELPDLPSSLLLPEDMALRNLPPLRAAHRRFNFDTDRPLLSTLEESVVRICCIRSFGHFIARLQGSILQFNPEVGIFVSIAQSEQESLLQQAQAQFRMAQEEARRNRLMRDMAQLRLQLEVSQLEGSLQQPKAQSAMSPYLVPDTQALCHHLPVIRQLATSGRFIVIIPRTVIDGLDLLKKEHPGARDGIRYLEAEFKKGNRYIRCQKEVGKSFERHKLKRQDADAWTLYKILDSCKQLTLAQGAGEEDPSGMVTIITGLPLDNPSVLSGPMQAALQAAAHASVDIKNVLDFYKQWKEIG

SEQ ID NO: 11 - DNA, 1536 bp, Homo sapiens
  source 1..1536: mol_type = genomic DNA
atggagacattccctgcagtggctgagaaggtcctcaaggagttccaggtgttactgcagcacagcccctctcccattggaagtacccgcatgctgcagcttatgaccatcaatatgtttgcagtacacaactcccagctgaaagactgcttctcggaggagtgccgctctgtgatccaggaacaagccgcagctctgggcttggccatgttttctctactggtccgccgctgcacctgcttacttaaggagtccgccaaagctcagctgtcctctcctgaggaccaggatgaccaagacgacatcaaggtgtcttcctttgtcccggacctgaaggagctgctccccagtgtcaaagtctggtcagattggatgctcggctacccggacacctggaatcctcctcccacatccctggatctgccctcgcatgttgctgtggatgtatggtcgacgctggctgatttctgtaacatactgactgcagtgaatcagtctgaggtgccactgtacaaggacccggatgatgacctcacccttcttatcctggaagaggatcggcttctctcgggctttgtccccttgctggctgcccctcaggacccctgctacgtggagaaaacctcggataaggttattgcagctgactgcaaaagggtcacagtgctgaagtattttctggaagccctttgtggacaagaagagcctctgctggcattcaagggtggaaagtatgtgtcagtggcacccgtcccagacaccatgggaaaggaaatgggaagccaagagggaacacgactggaagatgaggaggaggatgtggtgattgaagactttgaggaagattcagaggctgaaggcagcggaggcgaggatgacatcagggagcttcgggccaagaagctggctctggccaggaagatagctgagcagcagcgtcgccaggaaaagatccaggctgtcctggaggaccacagtcagatgaggcagatggagctcgaaatcagacctttgttcctcgtaccagacaccaacggcttcattgaccacctggccagtctggcgcggctgctggagagcaggaagtacatcctggtggtgcccctcatcgtgatcaatgagctggacggcctggccaaggggcaggagacagaccaccgggctgggggctacgcccgtgtggtacaagagaaggcccgcaagtccatcgagttcctcgagcagcgattcgagagtcgggactcttgcctgcgagccctgaccagccgtggcaatgaactcgaatccatcgccttccgcagtgaggacatcactggccagctgggtaacaacgatgatctcatcctgtcctgctgcctccactactgcaaagacaaggctaaggacttcatgcccgccagcaaagaggagccaatccggctactgcgggaggtggtgctgttgacggatgaccggaacctgcgtgtgaaggcgctcacaaggaatgttcctgtacgggacatcccagccttcctcacgtgggcccaggtgggctga

SEQ ID NO: 12 - protein, 511 aa, Homo sapiens
METFPAVAEKVLKEFQVLLQHSPSPIGSTRMLQLMTINMFAVHNSQLKDCFSEECRSVIQEQAAALGLAMFSLLVRRCTCLLKESAKAQLSSPEDQDDQDDIKVSSFVPDLKELLPSVKVWSDWMLGYPDTWNPPPTSLDLPSHVAVDVWSTLADFCNILTAVNQSEVPLYKDPDDDLTLLILEEDRLLSGFVPLLAAPQDPCYVEKTSDKVIAADCKRVTVLKYFLEALCGQEEPLLAFKGGKYVSVAPVPDTMGKEMGSQEGTRLEDEEEDVVIEDFEEDSEAEGSGGEDDIRELRAKKLALARKIAEQQRRQEKIQAVLEDHSQMRQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVG

SEQ ID NO: 13 - DNA, 3537 bp, Homo sapiens
  source 1..3537: mol_type = genomic DNA
atgagcctgcagagcgcgcagtacctccggcaggcagaagtcctgaaggctgacatgacagattctaagctgggtccagctgaagtctggacatccaggcaggctctgcaggacctgtaccagaaaatgctagttaccgatttggaatacgctttagacaagaaagtagaacaggatctctggaatcacgcctttaagaatcagatcacaacactacaaggccaggcaaagaatcgagcaaatccgaatcggagtgaagttcaggcaaacctttctctgttcctagaggcagctagtggcttctatactcagttattacaagaactgtgtacagtatttaatgtagatttaccatgccgtgtgaagtcttcccaattgggaattatcagcaataaacagacgcataccagcgccatagtgaagccacagtctagctcctgttcctatatctgccagcactgcctcgtccaccttggagacattgctcgatacagaaaccagaccagccaggcagagtcctactataggcatgcagctcagcttgtcccctccaatggtcagccttataatcagttggctatcttagcttcttccaaaggagaccatctgaccacaattttctactactgcagaagcattgctgtgaagttccctttcccagctgcctccactaatctgcaaaaagcactttctaaagcactggaaagccgagatgaggtgaaaaccaagtggggtgtttctgacttcatcaaggcctttattaaattccacggtcatgtgtacctgagtaagagcttggaaaagttgagccctcttcgagagaaattggaagaacagtttaagaggctgctattccaaaaagctttcaactctcagcagttagttcatgtcactgtcattaacctgtttcaacttcatcaccttcgtgactttagcaatgaaaccgagcagcacacttatagccaagatgagcagctatgttggacacagttgctggccctctttatgtcttttctcggcatcctgtgcaagtgtcctctacagaatgagtctcaggaggagtcctacaatgcctatcctcttccagcagtcaaggtctccatggactggctaagactcagacccagggtctttcaggaggcagtggtggatgaaagacagtacatttggccctggttgatttctcttctgaatagtttccatccccatgaagaggacctctcaagtattagtgcgacaccacttccagaggagtttgaattacaaggatttttggcattgagaccttctttcaggaacttggatttttccaaaggtcaccagggtattacaggggacaaagaaggccagcaacgacgaatacgacagcaacgcttgatctctataggcaaatggattgctgataatcagccaaggctgattcagtgtgaaaatgaggtagggaaattgttgtttatcacagaaatcccagaattaatactggaagaccccagtgaagccaaagagaacctcattctgcaagaaacatctgtgatagagtcgctggctgcagatgggagcccagggctaaaatcagtgctatctacaagccgaaatttaagcaacaactgtgacacaggagagaagccagtggttaccttcaaagaaaacattaagacacgagaagtgaacagagaccaaggaagaagttttcctcccaaagaggtaaaatcccagacagaactaagaaagactccagtgtctgaagccagaaaaacacctgtaactcaaaccccaactcaagcaagtaactcccagttcatccccattcatcaccctggagccttccctcctcttcccagcaggccagggtttccgcccccaacatatgttatccccccgcctgtggcattttctatgggctcaggttacaccttcccagctggtgtttctgtcccaggaacctttcttcagcctacagctcactctccagcaggaaaccaggtgcaagctgggaaacagtcccacattccttacagccagcaacggccctctggaccagggccaatgaaccagggacctcaacaatcacagccaccttcccagcaaccccttacatctttaccagctcagccaacagcacagtctacaagccagctgcaggttcaagctctaactcagcaacaacaatcccctacaaaagctgtgccggctttggggaaaagcccgcctcaccactctggattccagcagtatcaacaggcagatgcctccaaacagctgtggaatccccctcaggttcaaggcccattagggaaaattatgcctgtgaaacagccctactaccttcagacccaagaccccataaaactgtttgagccgtcattgcaacctcctgtaatgcagcagcagcctctagaaaaaaaaatgaagccttttcccatggagccatataaccataatccctcagaagtcaaggtcccagaattctactgggattcttcctacagcatggctgataacagatctgtaatggcacagcaagcaaacatagaccgcaggggcaaacggtcaccaggaatcttccgtccagagcaggatcctgtacccagaatgccgtttgaggaccccaagagctcccctctgcttcctccggacctgttaaagagtctggctgccttggaggaagaggaagagctgattttttctaacactcctgatctttacccggctctgctggggcctctcgcctctcttcctggacgaagcctttttaaatccttattggagaagccctcagagctcatgtcacattcatcctctttcctgtccctcaccggattctctctcaatcaggaaagatacccaaataatagtatgttcaatgaggtatatgggaaaaacctgacatccagctccaaagcagaactcagtccctcaatggccccccaggaaacatctctgtattccctttttgaagggactccgtggtctccatcacttcctgccagttcagatcattcaacaccagccagccagtctcctcattcctctaacccaagcagcctacccagctctcctccaacacacaaccataattctgttccattctccaattttggacccattgggactccagataacagggatagaaggactgcagatcggtggaaaactgataagccagccatgggtgggtttggcattgattatctctcagcaacgtcatcctctgagagcagttggcatcaggccagcactccgagtggcacctggacaggccatggcccttccatggaggattcctctgctgtcctcatggaaagcctaaagaagcaacagcatggggtccagcagttggggcccaaaagacagtctgaagaggaaggaagcagcagtatctgcgtagcccacagagggcccaggcccctgcccagctgcagtctcccagcctccactttcagagtgaaattcaaggcagcacggacatgtgcccatcaggcacagaagaaaacacgacgtcgtccattttggaagagacgaaagaaaggaaaataa

SEQ ID NO: 14 - protein, 1178 aa, Homo sapiens [sequence omitted]